(19) United States
(12) Patent Application Publication
(10) Pub. No.: US 2019/0116833 A1
(43) Pub. Date: Apr. 25, 2019

(54) FEED ADDITIVE COMPOSITIONS

(71) Applicant: NutriQuest, LLC, Mason City, IA (US)

(72) Inventors: Kim Friesen, Carthage, IN (US); Ran Song, Eden Prairie, MN (US); Chad Hagen, Sleepy Eye, MN (US)

(21) Appl. No.: 16/167,785

(22) Filed: Oct. 23, 2018

Related U.S. Application Data
(60) Provisional application No. 62/575,675, filed on Oct. 23, 2017.

(51) Int. Cl. A23K 20/22; A23K 20/174; A23K 10/16; A23K 50/60; A23K 50/30; A23K 50/10; A23K 50/40; A23K 50/75; A61K 36/064; A61K 31/51; A61K 33/04; A61K 9/00

(52) U.S. Cl. CPC A23K 20/22; A23K 20/174; A23K 10/16; A23K 50/60; A23K 50/30; A23K 50/10; A61P 1/00; A23K 50/75; A61K 36/064; A61K 31/51; A61K 33/04; A61K 9/0056; A23K 50/40

(57) ABSTRACT

The present invention provides feed additive composition comprising fermented yeast, sodium metabisulfite, and optionally thiamin. When a normal or basal feed is supplemented with the feed additive, improved growth, improved health, improved intestinal health, and a reduction of microbial pathogens is seen in non-human animals as compared to a control group.

### CROSS REFERENCE TO RELATED APPLICATIONS

The present application claims benefit under 35 U.S.C. § 119 of U.S. Provisional Patent Application No. 62/575,675, entitled, “FEED ADDITIVE COMPOSITIONS” filed Oct. 23, 2017, the entirety of which is incorporated herein by reference.

### FIELD OF THE INVENTION

The present disclosure generally relates to feed additive compositions, methods for improving growth, improving health, improving intestinal health, and the reduction of microbial pathogens in non-human animals.

### BACKGROUND OF THE INVENTION

Animal husbandry is a branch of agriculture concerned with raising, breeding, and day to day care of animals. Famers engaged in this branch of agriculture produce meat, eggs, milk, and other products for the consumer. Additionally, these farmers are engaged in breeding and raising a wide variety of animals.

Famers engaged in animal husbandry face many challenges. The demand for food and food products from animal husbandry is anticipated to increase significantly as the population is growing. Also, with the increase in population, increased demands on land, water, and energy resources are being realized. Global environmental challenges, including global climate changes, and the growing threat of disease transmission to and from agricultural animals adds further challenges. Therefore, farmers need to become more efficient, produce the products at a higher rate, and raise livestock and poultry at an increased rate to meet market challenges. Additionally, farmers need a low cost method to produce these products since the profit margin is these areas can be quite low.

In the past, farmers used antibiotics and hormones to not only control diseases and pathogens but also to promote growth in livestock and poultry. Due to changes in consumer sentiment towards the use of antibiotics, there is an increase of production that is reared without the use of antibiotics.

To meet these challenges, what is needed is a feed additive which is generally low cost but also promotes health, growth, and reduces the levels of pathogens in animals without having to use antibiotics and hormones.

### SUMMARY OF THE INVENTION

Provided herein are feed additive compositions useful for non-human animals, methods for improving growth improving health, improving intestinal health, and the reduction of microbial pathogens by supplementing a normal animal feed or basal feed with the feed additive composition wherein the supplemented feed is orally administered to the non-human animal at least once per day.

In one aspect, provided herein, are feed additive compositions comprising formulated yeast, sodium metabisulfite, and optionally thiamin.

Another aspect is methods improving growth in non-human animals. The method comprises supplementing normal animal feed or a basal feed with a feed additive composition and orally administrating the supplemented feed to non-human animals at least once per day.

An additional aspect is methods for improving health in non-human animals. The method comprises supplementing a normal animal feed or a basal feed with a feed additive composition and orally administrating the supplemented feed to non-human animals at least once per day.

Another aspect is methods for improving intestinal health and the reduction of microbial pathogens in non-human animals. The method comprises supplementing a normal animal feed or a basal feed with a feed additive composition and orally administrating the supplemented feed to the non-human animals at least once per day.

Other features and iterations of the invention are described in more detail below.

### DETAILED DESCRIPTION OF THE INVENTION

Disclosed herein are feed additive compositions useful for non-human animals. After the feed additive composition is added to normal animal feed or basal feed, oral administration to non-human animals at least once per day shows improved growth, improved health, improved intestinal health, and a reduction of microbial pathogens as compared to a control group not administered with the feed additive composition.

(I) Feed Additive Compositions

One aspect of the present disclosure encompasses feed additive compositions for non-human animals comprising formulated yeast, sodium metabisulfite, and optionally thiamin. Other optional additives may be further included. The feed additive composition may be formulated with normal animal feed or basal feed, or added to normal animal feed or basal feed before administration to the non-human animals.

(a) Formulated Yeast

As used herein, the formulated yeast product may comprise a combination of Saccharomyces cerevisiae yeast extract representing approximately 25-100% of the total formulated yeast product by weight, hydrolyzed yeast representing approximately 0-40% of the total formulated yeast product by weight, a yeast culture representing approximately 0-50% of the total formulated yeast product by weight. The formulated yeast may also comprise limestone representing approximately 0-50% of the total formulated yeast product by weight.

The formulated yeast may be any yeast provided the yeast is generally regarded as safe for use in food or medical applications. Non-limiting examples of formulated yeast-derived products may include yeast cell wall derived components such as β-glucans, arabinoxylan isomaltose, agarooligosaccharides, lactosucrose, cyclodextrins, lactose, fructooligosaccharides, laminariheptaose, lactulose, galactooligosaccharides, mannanoligosaccharides, raffinose, stachyose, oligofructose, glucosyl sucrose, sucrose thermal oligosaccharide, isomalturose, caramel, inulin, and xylooligosaccharides. In an embodiment, the formulated yeast may be β-glucans and/or mannanoligosaccharides. Sources for yeast cell wall derived components include Saccharomyces bisporus, Saccharomyces boulardii, Saccharomyces cerevisiae, Saccharomyces capsularis, Saccharomyces delbrueckii, Saccharomyces fermentati, Saccharomyces lugwigii, Saccharomyces microellipsoides, Saccharomyces pastorianus, Saccharomyces rosei, Candida albicans, Candida cloaceae, Candida tropicalis, Candida utilis, Geotrichum candidum, Hansenula americana, Hansenula anomala, Hansenula wingei, and Aspergillus oryzae.

The formulated yeast may also include bacteria cell wall derived agents such as peptidoglycan and other components derived from gram-positive bacteria with a high content of peptidoglycan. Exemplary gram-positive bacteria include Lactobacillus acidophilus, Bifedobact thermophilum, Bifedobat longhum, Streptococcus faecium, Bacillus pumilus, Bacillus subtilis, Bacillus licheniformis, Lactobacillus acidophilus, Lactobacillus casei, Enterococcus faecium, Bifidobacterium bifidium, Propionibacterium acidipropionici, Propionibacteriium freudenreichii, and Bifidobacterium pscudolongum.

A therapeutically effective amount of a formulated yeast in a feed composition can and will vary depending on the body weight, sex, and/or medical condition of the non-human animal, as well as the species of the non-human animal, and may be determined experimentally using methods known in the art. Generally, the amount of formulated yeast in the feed additive composition may range from about 0.5 lb/ton to 10.0 lb/ton. In various embodiments, the amount of formulated yeast in the feed additive composition may range from about 0.5 lb/ton to 10.0 lb/ton, from about 1.0 lb/ton to 9.0 lb/ton, from about 1.5 lb/ton to about 8.0 lb/ton, or from 2.0 lb/ton to about 7.0 lb/ton.

(b) Sodium Metabisulfite

The feed additive composition may further comprise sodium metabisulfite. The sodium metabisulfite, as appreciated by the skilled artisan, is known as a food additive and preservative. The addition of sodium metabisulfite to the feed additive provides a synergistic effect with the formulated yeast, as demonstrated below.

A therapeutic effective amount of the sodium metabisulfite can and will vary depending on the type of non-human animal, the body weight, sex, and medical condition of the non-human animal. Generally, the amount of sodium metabisulfite may range from about 1.0 lb/ton to 20 lb/ton. In various embodiments, the amount of sodium metabisulfite may range from about 1.0 lb/ton to 20 lb/ton, from about 1.5 lb/ton to 17.5 lb/ton, or from 2.0 lb/ton to 15 lb/ton.

(c) Thiamin

In an embodiment, the feed additive composition may further comprise thiamin, which is also referred to thiamine or vitamin B₁. Non-limiting examples of sources of thiamin may be Aneurine Hydrochloride, Antiberiberi Factor, Antiberiberi Vitamin, Antineuritic Factor, Antineuritic Vitamin, B Complex Vitamin, Chlorhydrate de Thiamine, Chlorure de Thiamine, Complexe de Vitamine B, Facteur Anti-béribéri, Facteur Antineuritique, Hydrochlorure de Thiamine, Mononitrate de Thiamine, Nitrate de Thiamine, Thiamine Chloride, Thiamine HCl, Thiamine Hydrochloride, Thiamin Mononitrate, Thiamine Mononitrate, Thiamine Nitrate, Thaimine diphosphate, Thiamine triphosphate, adenosine thiamine triphosphate, Thiaminium Chloride Hydrochloride, Tiamina, Vitamin B1, Vitamin B-1, Vitamina B1, Vitamine Anti-béribéri, Vitamine Antineuritique, and Vitamine B1.

In general, the amount of thiamin in the composition may range from about 0 ppm to 20 ppm. In various embodiments, the amount of thiamin in the composition may range from about 0 ppm to 20 ppm, from about 2 ppm to 15 pm, or from about 2 ppm to about 10 ppm.

(d) Normal Animal Feed/Basal Feed

“Normal feed” or “feed matter” as defined herein refers to a normal general feed provided to the non-human animals. “Basal feed” as defined herein refers to animal feeds which comprise concentrated sources of energy and are especially rich in starches and sugars. Basal feeds may include the whole group of grains (e.g. wheat, maize, oats, etc.) and their by-products. Generally, basal feeds generally have a protein content that is greater than 16% and a maximum fiber content of 18%. The main difference between basal feeds and other feed stuffs is that basal feeds have high digestible energy content.

The normal animal feed or basal feed may include one or more components. Non-limiting examples these components may include, without limitation: corn or a component of corn, such as, for example, corn meal, corn fiber, corn hulls, corn DDGS (distiller's dried grain with solubles), silage, ground corn, corn germ, corn gluten, corn oil, or any other portion of a corn plant; soy or a component of soy, such as, for example, soy oil, soy meal, soy hulls, soy silage, ground soy, or any other portion of a soy plant; wheat or any component of wheat, such as, for example, wheat meal, wheat fiber, wheat hulls, wheat chaff, ground wheat, wheat germ, or any other portion of a wheat plant; canola or any other portion of a canola plant, such as, for example, canola oil, canola meal, canola protein, canola hulls, ground canola, or any other portion of a canola plant; sunflower or a component of a sunflower plant; sorghum or a component of a sorghum plant; sugar beet or a component of a sugar beet plant; cane sugar or a component of a sugarcane plant; barley or a component of a barley plant; palm oil, palm kernel or a component of a palm plant; glycerol; corn steep liquor; a waste stream from an agricultural processing facility; lecithin; rumen protected fats; molasses; soy molasses; flax; peanuts; peas; oats; grasses, such as orchard grass and fescue; fish meal, meat & bone meal; feather meal; and poultry byproduct meal; and alfalfa and/or clover used for silage or hay, and various combinations of any of the feed ingredients set forth herein, or other feed ingredients generally known in the art. As it will be recognized in the art, a feed composition may further be supplemented with amino acids, vitamins, minerals, and other feed additives such as other types of enzymes, organic acids, essential oils, probiotics, prebiotics, antioxidants, pigments, anti-caking agents, and the like which are detailed below.

(i). Vitamins

Optionally, the normal animal feed or basal feed may include one or more vitamins. Suitable vitamins for use in the dietary supplement include vitamin C, vitamin A, vitamin E, vitamin B12, vitamin K, riboflavin, niacin, vitamin D, vitamin B6, folic acid, pyridoxine, thiamine, pantothenic acid, and biotin. The form of the vitamin may include salts of the vitamin, derivatives of the vitamin, compounds having the same or similar activity of a vitamin, and metabolites of a vitamin.

The normal animal feed or basal feed may include one or more forms of an effective amount of any of the vitamins described herein or otherwise known in the art. Exemplary vitamins include vitamin K, vitamin D, vitamin C, and biotin. An “effective amount” of a vitamin typically quantifies an amount at least about 10% of the United States Recommended Daily Allowance (“RDA”) of that particular vitamin for a subject. It is contemplated, however, that amounts of certain vitamins exceeding the RDA may be beneficial for certain animals. For example, the amount of a given vitamin may exceed the applicable RDA by 100%, 200%, 300%, 400%, 500% or more.

(ii) Minerals

Generally, the normal animal feed or basal feed may include one or more minerals or mineral sources. Non-limiting examples of minerals include, without limitation, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

Generally speaking, the normal animal feed or basal feed may include one or more forms of an effective amount of any of the minerals described herein or otherwise known in the art. An “effective amount” of a mineral typically quantifies an amount at least about 10% of the United States Recommended Daily Allowance (“RDA”) of that particular mineral for a subject. It is contemplated, however, that amounts of certain minerals exceeding the RDA may be beneficial for certain subjects. For example, the amount of a given mineral may exceed the applicable RDA by 100%, 200%, 300%, 400%, 500% or more. Typically, the amount of mineral included in the dietary supplement may range from about 1 mg to about 1500 mg, about 5 mg to about 500 mg, or from about 50 mg to about 500 mg per dosage.

(iii) Essential Fatty Acids

Optionally, the normal animal feed or basal feed may include a source of an essential fatty acid. The essential fatty acid may be isolated or it may be an oil source or fat source that contains an essential fatty acid. In one embodiment, the essential fatty acid may be a polyunsaturated fatty acid (PUFA), which has at least two carbon-carbon double bonds generally in the cis-configuration. The PUFA may be a long chain fatty acid having at least 18 carbons atoms. The PUFA may be an omega-3 fatty acid in which the first double bond occurs in the third carbon-carbon bond from the methyl end of the carbon chain (i.e., opposite the carboxyl acid group). Examples of omega-3 fatty acids include alpha-linolenic acid (18:3, ALA), stearidonic acid (18:4), eicosatetraenoic acid (20:4), eicosapentaenoic acid (20:5; EPA), docosatetraenoic acid (22:4), n-3 docosapentaenoic acid (22:5; n-3DPA), and docosahexaenoic acid (22:6; DHA). The PUFA may also be an omega-5 fatty acid, in which the first double bond occurs in the fifth carbon-carbon bond from the methyl end. Exemplary omega-5 fatty acids include myristoleic acid (14:1), myristoleic acid esters, and cetyl myristoleate. The PUFA may also be an omega-6 fatty acid, in which the first double bond occurs in the sixth carbon-carbon bond from the methyl end. Examples of omega-6 fatty acids include linoleic acid (18:2), gamma-linolenic acid (18:3), eicosadienoic acid (20:2), dihomo-gamma-linolenic acid (20:3), arachidonic acid (20:4), docosadienoic acid (22:2), adrenic acid (22:4), and n-6 docosapentaenoic acid (22:5). The fatty acid may also be an omega-9 fatty acid, such as oleic acid (18:1), eicosenoic acid (20:1), mead acid (20:3), erucic acid (22:1), and nervonic acid (24:1).

In another embodiment, the essential fatty acid source may be a seafood-derived oil. The seafood may be a vertebrate fish or a marine organism, such that the oil may be fish oil or marine oil. The long chain (20C, 22C) omega-3 and omega-6 fatty acids are found in seafood. The ratio of omega-3 to omega-6 fatty acids in seafood ranges from about 8:1 to 20:1. Seafood from which oil rich in omega-3 fatty acids may be derived include, but are not limited to, abalone scallops, albacore tuna, anchovies, catfish, clams, cod, gem fish, herring, lake trout, mackerel, menhaden, orange roughy, salmon, sardines, sea mullet, sea perch, shark, shrimp, squid, trout, and tuna.

In yet another embodiment, the essential fatty acid source may be a plant-derived oil. Plant and vegetable oils are rich in omega-6 fatty acids. Some plant-derived oils, such as flaxseed oil, are especially rich in omega-3 fatty acids. Plant or vegetable oils are generally extracted from the seeds of a plant, but may also be extracted from other parts of the plant. Plant or vegetable oils that are commonly used for cooking or flavoring include, but are not limited to, acai oil, almond oil, amaranth oil, apricot seed oil, argan oil, avocado seed oil, babassu oil, ben oil, blackcurrant seed oil, Borneo tallow nut oil, borage seed oil, buffalo gourd oil, canola oil, carob pod oil, cashew oil, castor oil, coconut oil, coriander seed oil, corn oil, cottonseed oil, evening primrose oil, false flax oil, flax seed oil, grapeseed oil, hazelnut oil, hemp seed oil, kapok seed oil, lallemantia oil, linseed oil, macadamia oil, meadowfoam seed oil, mustard seed oil, okra seed oil, olive oil, palm oil, palm kernel oil, peanut oil, pecan oil, pequi oil, perilla seed oil, pine nut oil, pistachio oil, poppy seed oil, prune kernel oil, pumpkin seed oil, quinoa oil, ramtil oil, rice bran oil, safflower oil, sesame oil, soybean oil, sunflower oil, tea oil, thistle oil, walnut oil, or wheat germ oil. The plant derived oil may also be hydrogenated or partially hydrogenated.

In still a further embodiment, the essential fatty acid source may be an algae-derived oil. Commercially available algae-derived oils include those from Crypthecodinium cohnii and Schizochytrium sp. Other suitable species of algae, from which oil is extracted, include Aphanizomenon flos-aquae, Bacilliarophy sp., Botryococcus braunii, Chlorophyceae sp., Dunaliella tertiolecta, Euglena gracilis, Isochrysis galbana, Nannochloropsis salina, Nannochloris sp., Neochloris oleoabundans, Phaeodactylum tricornutum, Pleurochrysis carterae, Prymnesium parvum, Scenedesmus dimorphus, Spirulina sp., and Tetraselmis chui.

(iv) Amino Acids

The normal animal feed or basal feed may optionally include from one to several amino acids. Suitable amino acids include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine or their hydroxy analogs. In certain embodiments, the amino acid will be selected from the essential amino acids. An essential amino acid is generally described as one that cannot be synthesized de novo by the organism, and therefore, must be provided in the diet. By way of non-limiting example, the essential amino acids for humans include: L-histidine, L-isoleucine, L-leucine, L-lysine, L-methionine, L-phenylalanine, L-valine and L-threonine.

(v) Antioxidants

The normal animal feed or basal feed may include one or more suitable antioxidants. As will be appreciated by a skilled artisan, the suitability of a given antioxidant will vary depending upon the species to which the dietary supplement will be administered. Non-limiting examples of antioxidants include ascorbic acid and its salts, ascorbyl palmitate, ascorbyl stearate, anoxomer, N-acetylcysteine, benzyl isothiocyanate, o-, m- or p-amino benzoic acid (o is anthranilic acid, p is PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, p-coumaric acid, curcurin, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eugenol, ferulic acid, flavonoids, flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palm ityl citrate, phenothiazine, phosphatidylcholine, phosphoric acid, phosphates, phytic acid, phytylubichromel, propyl gallate, polyphosphates, quercetin, trans-resveratrol, rosmarinic acid, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, zeaxanthin, or combinations thereof.

Natural antioxidants that may be included in the dietary supplement include, but are not limited to, apple peel extract, blueberry extract, carrot juice powder, clove extract, coffee berry, coffee bean extract, cranberry extract, eucalyptus extract, ginger powder, grape seed extract, green tea, olive leaf, parsley extract, peppermint, pimento extract, pomace, pomegranate extract, rice bran extract, rosehips, rosemary extract, sage extract, tart cherry extract, tomato extract, tumeric, and wheat germ oil.

(vi) Anti-Inflammatory Agents

The normal animal feed or basal feed may optionally include at least one anti-inflammatory agent. In one embodiment, the anti-inflammatory agent may be a synthetic non-steroidal anti-inflammatory drug (NSAID) such as acetylsalicylic acid, dichlophenac, indomethacin, oxamethacin, ibuprofen, indoprofen, naproxen, ketoprofen, mefamanic acid, metamizole, piroxicam, and celecoxib. In an alternate embodiment, the anti-inflammatory agent may be a prohormone that modulates inflammatory processes. Suitable prohormones having this property include prohormone convertase 1, proopiomelanocortin, prohormone B-type natriuretic peptide, SMR1 prohormone, and the like. In another embodiment, the anti-inflammatory agent may be an enzyme having anti-inflammatory effects. Examples of anti-inflammatory enzymes include bromelain, papain, serrapeptidase, and proteolytic enzymes such as pancreatin (a mixture of tyrpsin, amylase and lipase).

In still another embodiment, the anti-inflammatory agent may be a peptide with anti-inflammatory effects. For example, the peptide may be an inhibitor of phospholipase A2, such as antiflammin-1, a peptide that corresponds to amino acid residues 246-254 of lipocortin; antiflammin-2, a peptide that corresponds to amino acid residues 39-47 of uteroglobin; S7 peptide, which inhibits the interaction between interleukin 6 and interleukin 6 receptor; RP1, a prenyl protein inhibitor; and similar peptides. Alternatively, the anti-inflammatory peptide may be cortistatin, a cyclic neuropeptide related to somatostatin, or peptides that correspond to an N-terminal fragment of SV-IV protein, a conserved region of E-, L-, and P-selectins, and the like. Other suitable anti-inflammatory preparations include collagen hydrolysates and milk micronutrient concentrates (e.g., MicroLactin® available from Stolle Milk Biologics, Inc., Cincinnati, Ohio), as well as milk protein hydrolysates, casein hydrolysates, whey protein hydrolysates, and plant protein hydrolysates.

In a further embodiment, the anti-inflammatory agent may be a probiotic that has been shown to modulate inflammation. Suitable immunomodulatory probiotics include lactic acid bacteria such as acidophilli, lactobacilli, and bifidophilli. In yet another embodiment, the anti-inflammatory agent may be a plant extract having anti-inflammatory properties. Non-limiting examples of suitable plant extracts with anti-inflammatory benefits include blueberries, boswella, black catechu and Chinese skullcap, celery seed, chamomile, cherries, devils claw, eucalyptus, evening primrose, ginger, hawthorne berries, horsetail, Kalopanax pictus bark, licorice root, tumeric, white wallow, willow bark, and yucca.

(vii). Herbals

The normal animal feed or basal feed may optionally include at least one herb or herbal derivative. Suitable herbals and herbal derivatives, as used herein, refer to herbal extracts, and substances derived from plants and plant parts, such as leaves, flowers and roots, without limitation. Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, capsicum, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. John's wort, sarsaparilla, sassafras, saw palmetto, skullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yam, willow, wintergreen, witch hazel, wood betony, wormwood, yarrow, yellow dock, yerba santa, yucca and combinations thereof.

(viii). Pigments

The normal animal feed or basal feed may optionally include at least one pigment. Suitable non-limiting pigments include actinioerythrin, alizarin, alloxanthin, β-apo-2′-carotenal, apo-2-lycopenal, apo-6′-lycopenal, astacein, astaxanthin, azafrinaldehyde, aacterioruberin, aixin, α-carotine, β-carotine, γ-carotine, β-carotenone, canthaxanthin, capsanthin, capsorubin, citranaxanthin, citroxanthin, crocetin, crocetinsemialdehyde, crocin, crustaxanthin, cryptocapsin, α-cryptoxanthin, β-cryptoxanthin, cryptomonaxanthin, cynthiaxanthin, decaprenoxanthin, dehydroadonirubin, diadinoxanthin, 1,4-diamino-2,3-dihydroanthraquinone, 1,4-dihydroxyanthraquinone, 2,2′-diketospirilloxanthin, eschscholtzxanthin, eschscholtzxanthone, flexixanthin, foliachrome, fucoxanthin, gazaniaxanthin, hexahydrolycopene, hopkinsiaxanthin, hydroxyspheriodenone, isofucoxanthin, loroxanthin, lutein, luteoxanthin, lycopene, lycopersene, lycoxanthin, morindone, mutatoxanthin, neochrome, neoxanthin, nonaprenoxanthin, OH-Chlorobactene, okenone, oscillaxanthin, paracentrone, pectenolone, pectenoxanthin, peridinin, phleixanthophyll, phoeniconone, phoenicopterone, phoenicoxanthin, physalien, phytofluene, pyrrhoxanthininol, quinones, rhodopin, rhodopinal, rhodopinol, rhodovibrin, rhodoxanthin, rubixanthone, saproxanthin, semi-α-carotenone, semi-β-carotenone, sintaxanthin, siphonaxanthin, siphonein, spheroidene, tangeraxanthin, torularhodin, torularhodin methyl ester, torularhodinaldehyde, torulene, 1,2,4-trihydroxyanthraquinone, triphasiaxanthin, trollichrome, vaucheriaxanthin, violaxanthin, wamingone, xanthin, zeaxanthin, α-zeacarotene, or combinations thereof.

(ix). Pharmaceutical Acceptable Agents

The normal animal feed or basal feed may optionally include at least one pharmaceutical acceptable agent. Suitable non-limiting pharmaceutically acceptable agents include an acid/alkaline-labile drug, a pH dependent drug, or a drug that is a weak acid or a weak base. Examples of acid-labile drugs include statins (e.g., pravastatin, fluvastatin and atorvastatin), antiobiotics (e.g., penicillin G, ampicillin, streptomycin, erythromycin, clarithromycin and azithromycin), nucleoside analogs (e.g., dideoxyinosine (ddl or didanosine), dideoxyadenosine (ddA), dideoxycytosine (ddC)), salicylates (e.g., aspirin), digoxin, bupropion, pancreatin, midazolam, and methadone. Drugs that are only soluble at acid pH include nifedipine, emonapride, nicardipine, amosulalol, noscapine, propafenone, quinine, dipyridamole, josamycin, dilevalol, labetalol, enisoprost, and metronidazole. Drugs that are weak acids include phenobarbital, phenytoin, zidovudine (AZT), salicylates (e.g., aspirin), propionic acid compounds (e.g., ibuprofen), indole derivatives (e.g., indomethacin), fenamate compounds (e.g., meclofenamic acid), pyrrolealkanoic acid compounds (e.g., tolmetin), cephalosporins (e.g., cephalothin, cephalaxin, cefazolin, cephradine, cephapirin, cefamandole, and cefoxitin), 6-fluoroquinolones, and prostaglandins. Drugs that are weak bases include adrenergic agents (e.g., ephedrine, desoxyephedrine, phenylephrine, epinephrine, salbutamol, and terbutaline), cholinergic agents (e.g., physostigmine and neostigmine), antispasmodic agents (e.g., atropine, methantheline, and papaverine), curariform agents (e.g., chlorisondamine), tranquilizers and muscle relaxants (e.g., fluphenazine, thioridazine, trifluoperazine, chlorpromazine, and triflupromazine), antidepressants (e.g., am itriptyline and nortriptyline), antihistamines (e.g., diphenhydramine, chlorpheniramine, dimenhydrinate, tripelennamine, perphenazine, chlorprophenazine, and chlorprophenpyridamine), cardioactive agents (e.g., verapamil, diltiazem, gallapomil, cinnarizine, propranolol, metoprolol and nadolol), antimalarials (e.g., chloroquine), analgesics (e.g., propoxyphene and meperidine), antifungal agents (e.g., ketoconazole and itraconazole), antimicrobial agents (e.g., cefpodoxime, proxetil, and enoxacin), caffeine, theophylline, and morphine. In another embodiment, the drug may be a biphosphonate or another drug used to treat osteoporosis. Non-limiting examples of a biphosphonate include alendronate, ibandronate, risedronate, zoledronate, pamidronate, neridronate, olpadronate, etidronate, clodronate, and tiludronate. Other suitable drugs include estrogen, selective estrogen receptor modulators (SERMs), and parathyroid hormone (PTH) drugs. In yet another embodiment, the drug may be an antibacterial agent. Suitable antibiotics include aminoglycosides (e.g., amikacin, gentamicin, kanamycin, neomycin, netilmicin, streptomycin, and tobramycin), carbecephems (e.g., loracarbef), a carbapenem (e.g., certapenem, imipenem, and meropenem), cephalosporins (e.g., cefadroxil cefazolin, cephalexin, cefaclor, cefamandole, cephalexin, cefoxitin, cefprozil, cefuroxime, cefixime, cefdinir, cefditoren, cefoperazone, cefotaxime, cefpodoxime, ceftazidime, ceftibuten, ceftizoxime, and ceftriaxone), macrolides (e.g., azithromycin, clarithromycin, dirithromycin, erythromycin, and troleandomycin), monobactam, penicillins (e.g., amoxicillin, ampicillin, carbenicillin, cloxacillin, dicloxacillin, nafcillin, oxacillin, penicillin G, penicillin V, piperacillin, and ticarcillin), polypeptides (e.g., bacitracin, colistin, and polymyxin B), quinolones (e.g., ciprofloxacin, enoxacin, gatifloxacin, levofloxacin, lomefloxacin, moxifloxacin, norfloxacin, ofloxacin, and trovafloxacin), sulfonamides (e.g., mafenide, sulfacetamide, sulfamethizole, sulfasalazine, sulfisoxazole, and trimethoprim-sulfamethoxazole), and tetracyclines (e.g., demeclocycline, doxycycline, minocycline, and oxytetracycline). In an alternate embodiment, the drug may be an antiviral protease inhibitor (e.g., amprenavir, fosamprenavir, indinavir, lopinavir/ritonavir, ritonavir, saquinavir, and nelfinavir). In still another embodiment, the drug may be a cardiovascular drug. Examples of suitable cardiovascular agents include cardiotonic agents (e.g., digitalis (digoxin), ubidecarenone, and dopamine), vasodilating agents (e.g., nitroglycerin, captopril, dihydralazine, diltiazem, and isosorbide dinitrate), antihypertensive agents (e.g., alpha-methyldopa, chlortalidone, reserpine, syrosingopine, rescinnamine, prazosin, phentolamine, felodipine, propanolol, pindolol, labetalol, clonidine, captopril, enalapril, and lisonopril), beta blockers (e.g., levobunolol, pindolol, timolol maleate, bisoprolol, carvedilol, and butoxamine), alpha blockers (e.g., doxazosin, prazosin, phenoxybenzamine, phentolamine, tamsulosin, alfuzosin, and terazosin), calcium channel blockers (e.g., amlodipine, felodipine, nicardipine, nifedipine, nimodipine, nisoldipine, nitrendipine, lacidipine, lercanidipine, verapamil, gallopamil, and diltiazem), and anticlot agents (e.g., dipyrimadole).

(x). Excipients

A variety of commonly used excipients in normal animal feed or basal feed may be selected on the basis of compatibility with the active ingredients. Non-limiting examples of suitable excipients include an agent selected from the group consisting of non-effervescent disintegrants, a coloring agent, a flavor-modifying agent, an oral dispersing agent, a stabilizer, a preservative, a diluent, a compaction agent, a lubricant, a filler, a binder, taste masking agents, an effervescent disintegration agent, and combinations of any of these agents.

In one embodiment, the excipient is a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof. The polypeptide may be any arrangement of amino acids ranging from about 100 to about 300,000 daltons.

In another embodiment, the excipient may be a filler. Suitable fillers include carbohydrates, inorganic compounds, and polyvinylpirrolydone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol.

The excipient may comprise a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth.

In another embodiment, the excipient may be an effervescent disintegrant. By way of non-limiting example, suitable effervescent disintegrants include sodium bicarbonate in combination with citric acid and sodium bicarbonate in combination with tartaric acid.

The excipient may comprise a preservative. Suitable examples of preservatives include antioxidants, such as a-tocopherol or ascorbate, and antimicrobials, such as parabens, chlorobutanol or phenol.

In another embodiment, the excipient may include a diluent. Diluents suitable for use include pharmaceutically acceptable saccharide such as sucrose, dextrose, lactose, microcrystalline cellulose, fructose, xylitol, and sorbitol; polyhydric alcohols; a starch; pre-manufactured direct compression diluents; and mixtures of any of the foregoing.

The excipient may include flavors. Flavors incorporated into the outer layer may be chosen from synthetic flavor oils and flavoring aromatics and/or natural oils, extracts from plants, leaves, flowers, fruits, and combinations thereof. By way of example, these may include cinnamon oils, oil of wintergreen, peppermint oils, clover oil, hay oil, anise oil, eucalyptus, vanilla, citrus oil, such as lemon oil, orange oil, grape and grapefruit oil, fruit essences including apple, peach, pear, strawberry, raspberry, cherry, plum, pineapple, and apricot.

In another embodiment, the excipient may include a sweetener. By way of non-limiting example, the sweetener may be selected from glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, sylitol, and the like.

In another embodiment, the excipient may be a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

The excipient may be a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

Depending upon the embodiment, it may be desirable to provide a coloring agent in the outer layer. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in the present invention depending on the embodiment.

The excipient may include a taste-masking agent. Taste-masking materials include, e.g., cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, additional taste-masking materials contemplated are those described in U.S. Pat. Nos. 4,851,226, 5,075,114, and 5,876,759, each of which is hereby incorporated by reference in its entirety.

In various embodiments, the excipient may include a pH modifier. In certain embodiments, the pH modifier may include sodium carbonate or sodium bicarbonate.

Additionally, the antimicrobial clay may simply be added to any dosage form of a normal animal feed or basal feed.

The amount and types of ingredients (i.e., metal chelate, chondro protective agents, vitamin, mineral, amino acid, antioxidant, yeast culture, and essential fatty acid), and other excipients useful in normal animal feed or basal feed, are described throughout the specification and examples.

(e) Physical Form of the Feed Additive

According to various embodiments, the feed additive composition may be in any suitable form known in the animal feed art, and may be a wet or dry component. For example, according to certain embodiments, the feed additive composition may be in a form selected from the group consisting of a complete feed, a feed supplement, a feed additive, a premix, a top-dress, a tub, a mineral, a meal, a block, a pellet, a mash, a liquid supplement, a drench, a bolus, a treat, and combinations of any thereof. Additionally, a feed additive composition may optionally be ground before supplementing normal animal or basal feed.

(f) Introduction of the Feed Additive to Normal Animal Feed/Basal Feed

In various embodiments, the feed additive composition may be introduced to the normal animal feed or basal feed by way of various methods, depending on whether the feed additive composition is in a liquid or solid form. Non-limiting examples of introducing the feed additive composition to normal animal feed or basal feed may be formulating the feed additive composition into normal animal feed or basal feed, top-dressing the solid composition on normal animal feed or basal feed, spraying the liquid composition onto normal animal feed or basal feed, or combinations thereof.

(II) Methods for Improving the Growth of Non-Human Animals

Another aspect of the disclosure encompasses methods for improving the growth of non-human animals. The method comprises supplementing normal animal feed or basal feed with the feed additive composition as described in Section (I) and administering the supplemented normal animal feed or basal feed to non-human animals at least once a day. Non-human animals, in broad term, may be defined as an animal which exhibits improved growth after digestion of the supplemented normal or basal feed with the feed additive composition.

In various embodiments, the non-human animal may be a livestock mammal varying in age and health. Non-limiting example of suitable livestock mammals may be beef cattle, horses, dairy cattle, veal, pigs, goats, sheep, bison, llama, or alpaca. In other embodiments, the non-human animal may of avian species varying in age and health. Non-limiting examples of suitable avian species or poultry may be chickens, including broilers, layers, and breeders, ducks, game hens, geese, guinea fowl/hens, quail, and turkeys. In another embodiment, the non-human animal may be a companion animal varying in age and health. Non-limiting examples of companion animals may be a dog, a cat, a bird, a hamster, or a Guinee pig. In a preferred embodiment, the non-human animals is selected from a group comprising growing pigs, calves, foals, kids (goats), lambs, cria, chicks, poults, ducklings, puppies, kittens, or combinations thereof.

Preferably, when a feed additive composition is administered to non-human animals, a method of the invention comprises oral administration of a supplemented feed additive composition with normal animal feed or basal feed to the non-human animal. One or more doses of the feed additive composition with normal animal feed or basal feed may be administered to non-human animals. As will be appreciated by one of skill in the art, a dose of a feed additive composition can and will vary depending on the body weight, sex, age and/or medical condition of the non-human animals, the desired growth rate and efficiency desired, the microbial infection, the severity and extent of the microbial infection in the non-human animals, and the duration of treatment, as well as the species of the non-human animals.

“Improved growth,” as defined herein, refers to a positive change in size and/or maturation over a period of time in the non-human animal. In various embodiments, the non-human animals may exhibit improved growth including for example an increase in average daily weight gain (ADG), an increase in the average daily food intake (ADFI), an improved overall body weight, and the ratio F/G wherein the ratio of F/G is defined as the ADFI/ADG.

The non-human animals may exhibit an increase in the average daily weight gain (ADG) of at least 0.1 lbs as compared to a control group without supplementation of the feed additive composition. In various embodiments, the non-human animals may exhibit an increase in the average daily weight gain of at least 0.1 lbs, at least 0.2 lbs, at least 0.4 lbs, at least 0.5 lbs, at least 0.6 lbs, at least 0.7 lbs, at least 0.8 lbs, at least 0.9 lbs, at least 1.0 lbs, or more as compared to a control group without supplementation of the feed additive composition.

The non-human animals may exhibit an increase in the average daily food intake (ADFI) of at least of at least 0.1 lb/day as compared to a control group without supplementation of the feed additive composition. In various embodiments, the non-human animals may exhibit an increase in the average daily food intake of at least 0.1 lb/day, at least 0.2 lb/day, at least 0.3 lb/day, at least 0.4 lb/day, at least 0.5 lb/day, of at least 1.0 lb/day, at least 1.2 lb/day, at least 1.5 lb/day, or more as compared to a control group without supplementation of the feed additive composition.

The non-human animals may show an improved body weight as defined as the percent increase of at least 50% as compared to a control group without supplementation of the feed additive composition. In various embodiments, the percent increase of the improved body weight may be at least 50%, at least 75%, at least 100%, at least 200%, at least 300%, at least 400%, at least 500%, or more.

Another valuable measure to ascertain growth is the ratio F/G. The ratio F/G is defined as the ADFI/ADG. Generally, F/G may range from 1.0 to 2.0. In various embodiments, the F/G may range from 1.0 to 2.0, from 1.2 to 1.8, or from 1.4 to 1.6.

(III) Methods for Improving the Health of Non-Human Animals

Still another aspect of the disclosure encompasses methods for improving the health of non-human animals. The method comprises supplementing normal animal or basal feed with the feed additive composition as described in Section (I) and administering the supplemented normal or basal feed to non-human animals at least once a day. Non-human animals, in broad term, may be defined as an animal which exhibits improved health after digestion of the normal or basal feed supplemented with the feed additive.

In various embodiments, the non-human animal may be a livestock mammal varying in age and health. Non-limiting example of suitable livestock mammals may be beef cattle, horses, dairy cattle, veal, pigs, goats, sheep, bison, llama, or alpaca. In other embodiments, the non-human animal may of avian species varying in age and health. Non-limiting examples of suitable avian species or poultry may be chickens, including broilers, layers, and breeders, ducks, game hens, geese, guinea fowl/hens, quail, and turkeys. In another embodiment, the non-human animal may be a companion animal varying in age and health. Non-limiting examples of companion animals may be a dog, a cat, a bird a hamster, or a Guinee pig. In a preferred embodiment, the non-human animal is selected from a group comprising growing pigs, calves, foals, kids (goats), lambs, cria, chicks, poults, ducklings, puppies, kittens, or combinations thereof.

Preferably, when a feed additive composition is administered to non-human animals, a method of the invention comprises oral administration of a supplemented feed additive composition with normal animal feed or basal feed to the non-human animal. One or more doses of the feed additive composition with normal animal feed or basal feed may be administered to non-human animals. As will be appreciated by one of skill in the art, a dose of a feed additive composition can and will vary depending on the body weight, sex, age and/or medical condition of the non-human animals, the desired growth rate and efficiency desired, the microbial infection, the severity and extent of the microbial infection in the non-human animals, and the duration of treatment, as well as the species of the non-human animals.

“Improved health,” as defined herein, refers to a reduction of incidences of diarrhea, reduction in the number of days of diarrhea, a decrease in mortality, a decrease in cytokine panel measuring TNF-alpha, decrease in immunocrit levels, or combinations thereof in the non-human animals as compared to a control group.

(IV) Methods for Improving the Intestinal Health and the Reduction of Microbial Pathogens of Non-Human Animals

Still another aspect of the disclosure encompasses methods for improving the intestinal health and the reduction of microbial pathogens in the non-human animals. The method comprises supplementing normal animal or basal feed with the feed additive composition as described in Section (I) and administering the supplemented normal or basal feed to animals at least once a day to the non-human animal. Non-human animals, in broad term, may be defined as an animal which exhibits improving the intestinal health and the reduction of microbial pathogens after digestion of the normal animal or basal feed supplemented with the feed additive composition.

In various embodiments, the non-human animal may be a livestock mammal varying in age and health. Non-limiting example of suitable livestock mammals may be beef cattle, horses, dairy cattle, veal, pigs, goats, sheep, bison, llama, or alpaca. In other embodiments, the non-human animal may of avian species varying in age and health. Non-limiting examples of suitable avian species or poultry may be chickens, including broilers, layers, and breeders, ducks, game hens, geese, guinea fowl/hens, quail, and turkeys. In another embodiment, the non-human animal may be a companion animal varying in age and health. Non-limiting examples of companion animals may be a dog, a cat, a bird a hamster, or a Guinee pig. In a preferred embodiment, the non-human animals is selected from a group comprising growing pigs, calves, foals, kids (goats), lambs, cria, chicks, poults, ducklings, puppies, kittens, or combinations thereof.

Preferably, when a feed additive composition is administered to non-human animals, a method of the invention comprises oral administration of a supplemented feed additive composition with normal animal feed or basal feed to the non-human animal. One or more doses of the feed additive composition with normal animal feed or basal feed may be administered to non-human animals. As will be appreciated by one of skill in the art, a dose of a feed additive composition can and will vary depending on the body weight, sex, age and/or medical condition of the non-human animals, the desired growth rate and efficiency desired, the microbial infection, the severity and extent of the microbial infection in the non-human animals, and the duration of treatment, as well as the species of the non-human animals.

“Microbial pathogens,” as defined herein, refers to a micro-organism that has the potential to cause disease. An infection is the invasion and multiplication of pathogenic microbes in a subject. Disease is when the infection causes damage to the subject's vital functions or systems.

“Improved intestinal health” and the “reduction of microbial pathogens” refer to a reduction in the number of pathogens and a reduction of inflammation caused by the microbial pathogens in the non-human animal as compared to a control group. Non-limiting examples of pathogens may be E. Coli, E. Coli F18, E. Coli K88, salmonella, clostridium perfringens, Enterotoxigenic Escherichia coli (ETEC), or combinations thereof. The reduction of these pathogens is generally measured by a reduction in the serum kinase levels of IFN-α, IL-6, IL-8, IL-12, or combinations thereof.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms as used herein and in the claims shall include pluralities, and plural terms shall include the singular.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges can independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used herein, the terms “about” and “approximately” designate that a value is within a statistically meaningful range. Such a range can be typically within 20%, more typically still within 10%, and even more typically within 5% of a given value or range. The allowable variation encompassed by the terms “about” and “approximately” depends on the particular system under study and can be readily appreciated by one of ordinary skill in the art.

As used herein, “administering” is used in its broadest sense to mean contacting a non-human animal with a composition disclosed herein.

The phrases “therapeutically effective amount” and “antimicrobial effective amount” are used interchangeably to mean an amount that is intended to qualify the amount of an agent or compound, that when administered, it will achieve the goal of healing an infection site, inhibiting the growth of a microorganism, or otherwise benefiting the recipient environment.

As used herein, the terms “treating,” “treatment,” or “to treat” each may mean to alleviate, suppress, repress, eliminate, prevent or slow the appearance of symptoms, clinical signs, or underlying pathology of a condition or disorder on a temporary or permanent basis. Preventing a condition or disorder involves administering an agent of the present invention to a subject prior to onset of the condition. Suppressing a condition or disorder involves administering an agent of the present invention to a subject after induction of the condition or disorder but before its clinical appearance. Repressing the condition or disorder involves administering an agent of the present invention to a subject after clinical appearance of the disease. Prophylactic treatment may reduce the risk of developing the condition and/or lessen its severity if the condition later develops. For instance, treatment of a microbial infection may reduce, ameliorate, or altogether eliminate the infection, or prevent it from worsening.

As used herein, the term “w/w” designates the phrase “by weight” and is used to describe the concentration of a particular substance in a mixture or solution.

Having described the invention in detail, it will be apparent that modifications and variations are possible without departing from the scope of the invention defined in the appended claims.

### EXAMPLES

The following examples illustrate various embodiments of the invention.

### Example 1: Determine the Effect of Feeding Formulated Yeast to Pigs Challenged with F-18 E. coli

One hundred twenty weanling pigs (10.4±1.6 lbs; 18-21 d) were used to determine the effect of formulated yeast, direct fed microbials, and soluble fiber for reducing the impact of E. coli on growth performance and scours. Pigs were individually weighed at arrival (d −6), blocked by weight, and randomly allotted to one of 6 treatments (Table 1) resulting in 2 pigs per pen and 10 pens per treatment. Unchallenged pens were located away from the challenged pens with barriers around the unchallenged group. Barriers were placed between pens to assure that the challenge and responses to the challenge were contained to each individual pen.

On day −6, day 0 (prior to challenge), and day 3 pig weights were collected, and feed leftover was weighed by pen on day 0 and day 3 when pigs were weighed. Feed offered to each pen was recorded from day −6 to 3. This data was used to calculate pre- and post-challenge ADG, ADFI and G/F. Any removed pigs were recorded, along with BW, and date of removal.

Diets containing testing products were made by mixing the testing products into the basal diet (Table 2). Pigs were fed respective experimental diets from day −6 to 3. On day 0 and 1, each pig was orally inoculated with 5 ml (approximately 2.0×108/mL with a total challenge of 109 CFU) of the freshly grown E. coli F18 inoculants via a polyethylene tube attached to a syringe placed in the back of the oral cavity.

Each pig was monitored and assessed for occurrence and severity of post-weaning diarrhea using a fecal consistency scoring system (0=normal; 1=soft feces; 2=mild diarrhea; 3=severe diarrhea; Citation 1: Marquardt et al., 1999) at time 0 (prior to challenge), 1, 2, and 3-days post-challenge by the same trained personnel with no prior knowledge of dietary treatment allotment. Blood was collected at two time points: prior to challenge (day 0) and 3-dpi from 1 pig per pen (60 samples total). Initial bleed pig was recorded for the subsequent bleeding. One pig per pen was sacrificed from all treatment groups on day 3 of the trial for a total of 60 pigs. 15 cm of tied ileum tissue with digesta was collected beginning from the ileo-cecal junction to be quantified.

Data was analyzed using one-way ANOVA by the MIXED procedure of SAS for this complete randomized design. Pen was served as the experimental unit. The statistical model included fixed effect of dietary treatments and random effect of block. Initial pen BW was used as covariate for analysis of all responses. Multiple comparisons between treatments were performed using the Tukey adjustment option of SAS. All results were reported as least squares means. The significance level chosen was α=0.05. Treatment effect was considered significant if P<0.05, whereas values between 0.05≤P≤0.10 were considered as statistical trends.

TABLE 1
Experimental Treatments

| | Testing | Enteric | # Pigs per | | Total # |
|---|---|---|---|---|---|
| Treatment | Product | Challenge | Pen | # of Pens | Pigs |
| A | Non | None | 2 | 10 | 20 |
| | Challenge | | | | |
| B | Challenge | E. coli F18 | 2 | 10 | 20 |
| | Contr. | | | | |
| C | Formulated | E. coli F18 | 2 | 10 | 20 |
| | yeast ® | | | | |
| D | Formulated | E. coli F18 | 2 | 10 | 20 |
| | yeast ® + | | | | |
| | Product D | | | | |
| E | Formulated | E. coli F18 | 2 | 10 | 20 |
| | yeast ® + | | | | |
| | Product PSY | | | | |
| F | Formulated | E. coli F18 | 2 | 10 | 20 |
| | yeast ® + | | | | |
| | Product VW | | | | |

TABLE 2
Composition of the Basal Diet.

| Ingredient | Lbs/ton |
|---|---|
| Corn 600 14% Moisture | 860.26 |
| Whey dried | 417.00 |
| Soybean 46% | 350.00 |
| Hamlet HP300 | 150.00 |
| Menhaden SS-Fish Meal | 106.00 |
| Pork Fat | 67.00 |
| 21% Monocal | 14.00 |
| Salt | 11.00 |
| Limestone | 9.10 |
| Lysine 98.5 | 5.47 |
| Methionine 99% DL | 3.49 |
| Threonine 98.5% | 2.18 |
| Choline Chloride 60% | 1.00 |
| Swine trace mineral | 1.50 |
| Vitamin Premix | 1.00 |
| Sel-Plex 600 ppm | 0.50 |
| Selenium 0.06% | 0.50 |
| Total | 2000.00 |

Calculated Nutrient Concentration

| | |
|---|---|
| Crude Protein, % | 21.12 |
| Fat, % | 5.7 |
| Fiber, % | 1.98 |
| ME, kcal/lb | 1531.45 |
| Lysine, % | 1.52 |
| SW SI dig Lys, % | 1.36 |
| TID Thr/Ly, % | 0.65 |
| TID M + C/Ly, % | 0.57 |
| TID Try/Ly, % | 0.17 |
| TID Iso/Ly, % | 0.61 |
| TID Val/Ly, % | 0.67 |
| Calcium, % | 0.90 |
| Phosphorus, % | 0.75 |
| Ca/P Ratio, ratio | 1.2 |
| SW dig P, % | 0.52 |
| Lactose, % | 15.01 |

Pigs fed formulated yeast had reduced total E. coli, ETEC and F-18 counts compared the control pigs (Table 3). The reduction in bacterial load resulted in reduced diarrhea frequency. Additionally, the challenged pigs had improved feed intake and average daily gain, suggesting that the formulated yeast was effective in controlling the E. coli disease challenge.

TABLE 3
Summary 2017-ETEC3 Formulated Yeast Formulation ETEC Study

| | | | | | | | | |
|---|---|---|---|---|---|---|---|---|
| | | | | Formulated | | | | |
| | Non- | | | Yeast + | Formulated + | Formulated | | |
| | Challenged | | Formulated | Product | Product | Yeast + | | P- |
| Item | Control | Control | Yeast | D | PSY | VW | PSE | Value |

Treatment

| | | | | | | | | |
|---|---|---|---|---|---|---|---|---|
| # of Pens | 10 | 10 | 10 | 10 | 10 | 10 | N/A | N/A |
| # of Pigs | 20 | 20 | 20 | 20 | 20 | 20 | N/A | N/A |
| Start | 10.4 | 10.4 | 10.4 | 10.4 | 10.4 | 10.4 | 0.5 | 1.00 |
| BW,² lb | | | | | | | | |

Prior to Challenge

| | | | | | | | | |
|---|---|---|---|---|---|---|---|---|
| ADG, lb/day | 0.08 | 0.06 | −0.02 | 0.10 | 0.06 | 0.12 | 0.04 | 0.35 |
| ADFI, lb/day | 0.19 | 0.22 | 0.17 | 0.22 | 0.21 | 0.25 | 0.02 | 0.29 |
| BW on the | 10.9 | 10.8 | 10.3 | 11.0 | 10.7 | 11.1 | 0.3 | 0.35 |
| day of | | | | | | | | |
| challenge, | | | | | | | | |
| lb | | | | | | | | |

Post Challenge

| | | | | | | | | |
|---|---|---|---|---|---|---|---|---|
| ADG, lb/day | 0.24^{a} | −0.39^{b} | −0.01^{ab} | −0.29^{b} | −0.28^{b} | 0.01^{ab} | 0.11 | 0.002 |
| ADFI, lb/day | 0.38 | 0.27 | 0.34 | 0.39 | 0.33 | 0.30 | 0.04 | 0.22 |
| Final BW, lb | 11.6^{a} | 9.9^{c} | 10.5^{abc} | 10.6^{abc} | 10.1^{bc} | 11.2^{ab} | 0.4 | 0.009 |
| Mortality, % | 0^{a} | 20^{b} | 20^{b} | 20^{b} | 20^{b} | 10^{ab} | N/A | 0.46 |

Fecal Score³

| | | | | | | | | |
|---|---|---|---|---|---|---|---|---|
| 0-dpi | 0.8 | 1.1 | 0.7 | 1.1 | 1.3 | 0.9 | 0.2851 | 0.67 |
| (prior to | | | | | | | | |
| challenge) | | | | | | | | |
| 1-dpi | 0.8^{x} | 1.9^{y} | 1.1^{xy} | 1.7^{xy} | 1.8^{xy} | 1.6^{xy} | 0.2891 | 0.06 |
| 2-dpi | 0.9^{x} | 2.1^{y} | 1.3^{xy} | 1.2^{xy} | 1.5^{xy} | 1.3^{xy} | 0.2862 | 0.13 |
| 3-dpi | 0.6^{a} | 1.8^{b} | 0.9^{ab} | 1.0^{ab} | 1.9^{b} | 1.1^{ab} | 0.3053 | 0.02 |
| Average | 0.8^{a} | 2.0^{b} | 1.3^{ab} | 1.5^{ab} | 1.7^{b} | 1.4^{ab} | 0.2172 | 0.006 |
| Diarrhea | 32^{a} | 76^{c} | 37^{a} | 47^{a} | 60^{bc} | 53^{abc} | N/A | 0.007 |
| frequency,⁴ % | | | | | | | | |

Bacteria Count in Ileum Tissue

| | | | | | | | | |
|---|---|---|---|---|---|---|---|---|
| E. coli, | 6.9^{ab} | 7.7^{a} | 6.5^{b} | 7.0^{ab} | 7.3^{ab} | 7.1^{ab} | 0.4 | 0.10 |
| log cfu/g | | | | | | | | |
| ETEC, log | 6.8^{ab} | 7.6^{a} | 6.0^{b} | 6.9^{ab} | 7.2^{ab} | 6.9^{ab} | 0.4 | 0.10 |
| cfu/g | | | | | | | | |
| F18, log | 6.8^{x} | 5.8^{xy} | 3.2^{y} | 5.3^{xy} | 3.9^{xy} | 4.8^{xy} | 1.0 | 0.11 |
| cfu/g | | | | | | | | |

Post Challenge Serum Cytokines⁵

| | | | | | | | | |
|---|---|---|---|---|---|---|---|---|
| IFN-α, pg/mL | 4.8 | 4.4 | 4.0 | 2.3 | 2.7 | 3.0 | 1.7 | 0.30 |
| IL-8, pg/mL | 215 | 294 | 169 | 230 | 149 | 167 | 89 | 0.71 |
| IL-12, pg/mL | 508 | 615 | 339 | 438 | 469 | 501 | 236 | 0.80 |

¹Formulated yeast was included at 2.0 lb/ton.
²Start BW was used as covariate in all analyses
³Fecal score was measured on a 0 to 3 scale: 0 = no signs of diarrhea; 3 = most severe signs of diarrhea
⁴Diarrhea frequency = number of days that pigs with fecal score >= 2/total pig days
⁵Values of serum cytokines on the challenge date were used as covariate
^{abc}Means without a common superscript within a row differ (P < 0.05)
^{xy}Means without a common superscript within a row tend to differ (P < 0.10)

### Example 2: Effect of Feeding Formulated Yeast and Sodium Meta-Bisulfate (SMBS) to Weanling Pigs Challenged with E. coli F18

120 weanling pigs (10-16 lb; 18-21 d) were weighed and randomly allotted to one of 6 dietary treatments (Table 4) resulting in 2 pigs per pen and 10 pens per treatment. Diets containing testing products were made by mixing the testing products into the basal diet (Table 2). Pigs were fed respective experimental diets from d −7 to 3-dpi or for 10 total days on test. The challenged pigs were inoculated on d 0 and 1-dpi, respectively with 5 ml (approximately 2.0×108/mL with a total challenge of 109 CFU) of the freshly grown E. coli F18 inoculants via a polyethylene tube attached to a syringe placed in the back of the oral cavity.

Pigs were individually weighed on d −7 (at placement), d 0 (prior to challenge), and 3-dpi (end of the trial). Feed leftovers were taken with the dry feeder weight and weighed following pig weights on weigh days. Feed offered to each pen was recorded from d −7 to 3, and used pre- and post-challenge ADG, ADFI and G:F.

Each pig was monitored and assessed for occurrence and severity of post-weaning diarrhea using a fecal consistency scoring system (0=normal; 1=soft feces; 2=mild diarrhea; 3=severe diarrhea; Citation 1: Marquardt et al., 1999) for four times: at time 0 (prior to challenge), 1-dpi, 2-dpi, 3-dpi, and by the same trained personnel with no prior knowledge of dietary treatment allotment aside from the non-challenge group.

Blood samples were collected prior to challenge and 3-dpi from 1 pig per pen (60 samples total) to determine changes in inflammatory cytokines. At each collection, blood will be collected by venipuncture from the anterior vena cava into 8.5 mL serum tubes with 20 gauge by 1 inch needles. One pig per pen will be sacrificed from all treatment groups and 15 cm of tied ileum tissue (with digesta), approximately 1 inch from the ileo-cecal junction was analyzed for E. coli and F18 counts.

TABLE 4
Dietary Treatments

| | Testing | Enteric | # Pigs per | | |
|---|---|---|---|---|---|
| Treatment | Product | Challenge | Pen | # of Pens | Total # Pigs |
| A | Non | None | 2 | 10 | 20 |
| | Challenge | | | | |
| B | Challenge | E. coli F18 | 2 | 10 | 20 |
| | Control | | | | |
| C | Formulated | E. coli F18 | 2 | 10 | 20 |
| | yeast ® | | | | |
| D | Formulated | E. coli F18 | 2 | 10 | 20 |
| | yeast ® + | | | | |
| | SMBS | | | | |
| E | Formulated | E. coli F18 | 2 | 10 | 20 |
| | yeast ® + | | | | |
| | SMBS | | | | |
| F | Formulated | E. coli F18 | 2 | 10 | 20 |
| | yeast ® + | | | | |
| | SMBS | | | | |

The data from this study suggest that formulated yeast alone and the combination of formulated yeast and sodium meta-bisulfite have positive impacts on growth performance and controlling E. coli and disease challenged pigs (Table 5).

TABLE 5
Summary 2017-ETEC4 Formulated Yeast Formulation ETEC Study

| | | | | | | | | |
|---|---|---|---|---|---|---|---|---|
| | Non- | | | Yeast + | Yeast + | Yeast + | | |
| | Challenged | | | SMBS − | SMBS − | SMBS − | | P- |
| Item | Control | Control | Yeast | 5 lb/ton | 10 lb/ton | 15 lb/ton | PSE | value |

Treatment

| | | | | | | | | |
|---|---|---|---|---|---|---|---|---|
| # of Pens | 10 | 10 | 10 | 10 | 10 | 10 | | |
| # of Pigs | 20 | 20 | 20 | 20 | 20 | 20 | | |
| Start BW,² lb | 11.0 | 10.9 | 11.0 | 11.0 | 11.0 | 11.0 | 0.3 | 0.30 |

Prior to challenge

| | | | | | | | | |
|---|---|---|---|---|---|---|---|---|
| ADG, lb/day | 0.24 | 0.22 | 0.37 | 0.27 | 0.42 | 0.41 | 0.06 | 0.05 |
| ADFI, lb/day | 0.33 | 0.29 | 0.39 | 0.31 | 0.40 | 0.41 | 0.04 | 0.08 |
| BW on the | 12.7 | 12.5 | 13.6 | 12.9 | 13.9 | 13.8 | 0.40 | 0.05 |
| day of | | | | | | | | |
| challenge, | | | | | | | | |
| lb | | | | | | | | |

Post challenge

| | | | | | | | | |
|---|---|---|---|---|---|---|---|---|
| ADG, lb/day | 0.68^{a} | 0.00^{b} | 0.35^{ab} | 0.14^{b} | 0.10^{b} | −0.04^{b} | 0.11 | 0.004 |
| ADFI, lb/day | 0.68^{ab} | 0.46^{a} | 0.72^{b} | 0.48^{a} | 0.62^{ab} | 0.59^{ab} | 0.06 | 0.02 |
| Final BW, lb | 14.7^{xy} | 12.8^{x} | 14.7^{y} | 13.4^{xy} | 14.2^{xy} | 13.8^{xy} | 0.52 | 0.06 |
| Mortality, % | 0.0 | 5.0 | 0.0 | 0.0 | 5.0 | 10.0 | | 0.004 |

Fecal score³

| | | | | | | | | |
|---|---|---|---|---|---|---|---|---|
| 0-dpi | 1.2 | 1.4 | 1.3 | 1.2 | 0.7 | 1.3 | 0.3 | 0.57 |
| (prior to | | | | | | | | |
| challenge) | | | | | | | | |
| 1-dpi | 1.1 | 1.1 | 0.6 | 1.0 | 0.9 | 1.3 | 0.2 | 0.32 |
| 2-dpi | 0.9 | 1.0 | 0.6 | 1.0 | 0.8 | 1.0 | 0.3 | 0.86 |
| 3-dpi | 0.7 | 1.3 | 0.8 | 1.1 | 1.0 | 1.0 | 0.2 | 0.33 |
| Average | 0.9 | 1.2 | 0.6 | 1.0 | 0.9 | 1.2 | 0.2 | 0.28 |
| Diarrhea | 33 | 42 | 22 | 38 | 37 | 39 | | 0.44 |
| frequency,⁴ % | | | | | | | | |

Bacteria count in ileum tissue

| | | | | | | | | |
|---|---|---|---|---|---|---|---|---|
| E coli., | 4.8 | 6.4 | 5.7 | 6.5 | 6.3 | 6.4 | 0.5 | 0.11 |
| log cfu/g | | | | | | | | |
| ETEC, log | 3.8^{a} | 6.3^{b} | 5.6^{ab} | 6.5^{b} | 6.0^{b} | 6.0^{b} | 0.6 | 0.02 |
| cfu/g | | | | | | | | |
| F18, log | 2.4^{x} | 1.2^{xy} | 0.5^{xy} | 2.0^{xy} | 0.5^{xy} | 0.0^{y} | 0.6 | 0.08 |
| cfu/g | | | | | | | | |

¹Formulated yeast was included at 2.0 lb/ton; SMBS was included at 5, 10, and 15 lb/ton, respectively, for each treatment.
²Start BW was used as covariate in all analyses
³Fecal score was measured on a 0 to 3 scale: 0 = no signs of diarrhea; 3 = most severe signs of diarrhea
⁴Diarrhea frequency = number of days that pigs with fecal score >= 2/total pig days
^{ab}Means without a common superscript within a row differ (P < 0.05)
^{xy}Means without a common superscript within a row tend to differ (P < 0.10)

### Example 3: Effect of Sodium Meta-Bisulfite on Growth Performance of Early Weaned Pigs, SMBS Titration

(a) Linear increase in ADG when titrating from 0 to 10 lb/ton

(b) Linear decrease in F/G when titrating from 0 to 10 lb/ton.

A total of 1,296 weanling pigs were used to determine the impact of sodium meta-bisulfate on growth performance in 19 to 20 day old pigs. The ratio of same-sow source pens was equalized within treatments. The number of barrows and gilts was balanced within the pen (either 13 barrows or 14 barrows per pen). Upon arrival weanling pigs were sorted to pens (48 pens with 27 pigs per pen) and weighed as a complete pen of pigs. Pens within block were randomly assigned to one of 4 dietary treatments (1 control and 3 experimental diets; Table 6) in a randomized complete block design. Diets containing testing products were made by mixing the testing products into the basal diet (Table 7).

Pig weights were taken at placement and at the end of each diet phase (day 9 and 23). Feed weights were collected daily and the data was used to calculate ADG, ADFI and F/G for both diet phases and for the entire experimental period (23 days). Blood samples were on day 0 and 23 of the study from one barrow in each pen. Each pen was selected at the beginning of the study, identified with an ear tag, and sampled again at the end of the study. 8.5 mL blood was collected and allowed to clot on ice for at least 1.5 hr and centrifuged at 2,000×g for 13 min (4° C.). Fecal scores for each pen were collected on d 3, 5, 7, and 10 using the following scale: 0=none; 1=mild; 2=substantial; 3=severe (Citation 1: Marquardt et al., 1999).

TABLE 6
Dietary Treatment

| | | Inclusion | | | Total # of |
|---|---|---|---|---|---|
| Treatment | Additive | Rate | # of Pens | # Pigs/Pen | Pigs |
| 1. Control | None | | 12 | 27 | 324 |
| 2. | SMBS + 2.5 | 2.5 lb/ton | 12 | 27 | 324 |
| | | SMBS +2 | | | |
| | | ppm | | | |
| | | thiamine¹ | | | |
| 3. | SMBS + 5.0 | 5.0 lb/ton | 12 | 27 | 324 |
| | | SMBS +2 | | | |
| | | ppm | | | |
| | | thiamine¹ | | | |
| 4. | SMBS + 10.0 | 10.0 lb/ton | 12 | 27 | 324 |
| | | SMBS +2 | | | |
| | | ppm | | | |
| | | thiamine¹ | | | |
| | | | | Total | 1296 |

TABLE 7
Composition of Basal Diet

| Ingredients | RES 9-15 | RES 15-25 |
|---|---|---|
| corn 600 14% moisture | 648.90 | 881.04 |
| Soybean meal 46.2% | 200.00 | 432.00 |
| Crumble Basemix NHF | 951.09 | 475.00 |
| Premix¹ | | |
| DOGS-Aurora SD | 100.00 | 100.00 |
| Choice White Grease | 30.00 | 40.00 |
| betaGRO | 6.00 | 3.00 |
| Lysine 98.5 | 6.50 | 7.40 |
| Methionine 99% DL | 3.75 | 3.50 |
| Threonine 98.5% | 2.55 | 2.80 |
| Tryptophan 98.5% | 0.71 | 0.56 |
| 21% Monocal | 10.10 | 19.90 |
| Limestone | 2.10 | 8.00 |
| Salt | 7.00 | 7.60 |
| Nursery VTM 3# | 3.00 | 3.00 |
| Optiphos 2000 | N/A | N/A |
| TBCC (Intellibond C) | N/A | N/A |
| ZnO 72% | 8.30 | 8.30 |
| Mecadox 2.5 | 20.0 | N/A |
| Aureomycin 90 (CTC) | N/A | 4.40 |
| Denagard 10 | N/A | 3.50 |
| Total Batch | 2000.00 | 2000.00 |
| Mod ME, kcal/lb | 1514 | 1502 |
| SID Lysine, g/mcal | 4.19 | 4.08 |
| SID Thr/Ly, %A | 0.63 | 0.62 |
| SID M + C/Lys, % | 0.58 | 0.58 |
| SID Try/Lys, % | 0.19 | 0.19 |
| SID Iso/Lys, % | 0.60 | 0.60 |
| SID Val/Lys, % | 0.65 | 0.65 |
| Protein, % | 23.31 | 22.66 |
| Lysine, % | 1.57 | 1.52 |
| SID Lysin, % e | 1.40 | 1.35 |
| Avail Phos Equil, % | 0.50 | 0.46 |
| Calcium, % | 0.67 | 0.69 |
| Anzl Calcium, % | 0.66 | 0.69 |
| Phosphorus, % | 0.74 | 0.74 |
| Lactose, % | 15.01 | 7.50 |
| Sodium, % | 0.41 | 0.30 |

¹Crumble packs include spray dried whey (877 lb/ton), dehulled SBM (316 lb/ton), HP 300 (316 lb/ton), corn-fine (229 lb/ton), fish meal (200 lb/ton), and pork fat (62 lb/ton).

Pigs fed increasing levels of sodium meta-bisulfate had linear (P<0.05) improvements in ADG and F/G in both phases of the study (Table 8). These data indicate that performance can be enhanced in non-challenged pigs. Improvements in fecal scores suggest, reduced potential for diarrhea indicating a potential for improved intestinal health which will manifest itself as improved growth performance.

TABLE 8
Summary 2017-12N SMBS Nursery Titrations

| | | | | | | | | |
|---|---|---|---|---|---|---|---|---|
| Item | Control | SMBS_2.5 | SMBS_5.0 | SMBS_10.0 | PSE | P-overall | P-linear | P-quadratic |

Treatment

| | | | | | | | | |
|---|---|---|---|---|---|---|---|---|
| # of Pens | 12 | 12 | 12 | 12 | | | | |
| # of Pigs | 324 | 324 | 324 | 324 | | | | |
| Start | 11.0 | 10.9 | 11.0 | 10.9 | 0.1 | 0.91 | 1.00 | 0.99 |
| BW,² | | | | | | | | |
| lb | | | | | | | | |

Phase 1; 9 days

| | | | | | | | | |
|---|---|---|---|---|---|---|---|---|
| ADG, lb/day | 0.12^{a} | 0.10^{a} | 0.12^{a} | 0.19^{b} | 0.02 | <.0001 | 0.004 | 0.02 |
| ADFI, lb/day | 0.54 | 0.54 | 0.53 | 0.56 | 0.01 | 0.13 | 0.37 | 0.13 |
| G:F | 0.21^{a} | 0.18^{a} | 0.22^{a} | 0.34^{b} | 0.03 | <.0001 | 0.003 | 0.03 |
| BW end of | 12.0^{a} | 11.9^{a} | 12.1^{a} | 12.6^{b} | 0.2 | <.0001 | 0.003 | 0.06 |
| Phase 1, lb | | | | | | | | |

Phase 2; 14 days

| | | | | | | | | |
|---|---|---|---|---|---|---|---|---|
| ADG, lb/day | 0.66^{a} | 0.70^{b} | 0.71^{b} | 0.75^{c} | 0.01 | <.0001 | <.0001 | 0.92 |
| ADFI, lb/day | 0.92^{a} | 0.96^{a} | 0.98^{b} | 1.01^{bc} | 0.02 | 0.002 | 0.0006 | 0.94 |
| F:G | 1.39 | 1.36 | 1.39 | 1.35 | 0.02 | 0.19 | 0.14 | 0.96 |
| BW end of | 21.3^{a} | 21.9^{a} | 22.1^{b} | 23.2^{b} | 0.3 | <.0001 | <.0001 | 0.39 |
| Phase 2, lb | | | | | | | | |

Phase 1 + 2; 23 days

| | | | | | | | | |
|---|---|---|---|---|---|---|---|---|
| ADG, lb/day | 0.44^{a} | 0.46^{a} | 0.47^{a} | 0.53^{b} | 0.01 | <.0001 | <.0001 | 0.11 |
| ADFI, lb/day | 0.77^{a} | 0.79^{a} | 0.80^{ab} | 0.83^{b} | 0.01 | 0.004 | 0.002 | 0.50 |
| F:G | 1.74^{a} | 1.72^{a} | 1.71^{a} | 1.58^{b} | 0.03 | <.0001 | <.0001 | 0.05 |
| Removal, % | 4.0^{ab} | 7.1^{a} | 6.8^{a} | 3.1^{b} | | 0.06 | | |

Fecal score

| | | | | | | | | |
|---|---|---|---|---|---|---|---|---|
| Day 3 | 0.46 | 0.54 | 0.63 | 0.46 | 0.14 | 0.71 | 0.89 | 0.34 |
| Day 5 | 1.23 | 1.05 | 1.23 | 1.22 | 0.17 | 0.72 | 0.80 | 0.57 |
| Day 7 | 1.50 | 1.17 | 1.24 | 1.33 | 0.14 | 0.16 | 0.45 | 0.09 |
| Average | 1.05 | 0.94 | 1.03 | 1.00 | 0.09 | 0.73 | 0.91 | 0.61 |

¹Control = No SMBS; SMBS_2.5 = SMBS at 2.5 lb/ton of complete feed; SMBS_5.0 = SMBS at 5.0 lb/ton of complete feed SMBS_10.0 = SMBS at 10.0 lb/ton of complete feed
²Start BW at weaning was used as covariate in analyses of growth performance data
^{abc}Means without a common superscript within a row differ (P < 0.05)
^{xy}Means without a common superscript within a row tend to differ (P < 0.10)

### Example 4: Effect of Combining Formulated Yeast with Sodium Meta-Bisulfite on Weaned Pig Growth Performance

A total of 2,592 weanling pigs were used to determine the impact of formulated yeast and sodium meta-bisulfite on growth performance in weaned pigs. On arrive pigs were weighted and sorted into pens (96 pens, 27 pigs/pen) containing equal numbers of gilts and barrows in each pen. Pens were ranked and blocked by weight, and within each block the pen was randomly assigned to one of 6 dietary treatments (1 control and 5 experimental diets; Table 9) in a randomized complete block design (16 pens/block). Diets containing testing products were made by mixing the testing products into the basal diet (Table 10).

The experiment was conducted for 52 days with a 4-phase feeding program (Table 11), with feed provided through the FeedLogic® system allowing collection of feed intake data by pen. Pig weights were collected on apen basis upon arrival, and at each diet change.

Blood samples were collected at three time points: day 0, end of Phase 2, and Phase 4 from one barrow/pen (average size). At placement, one average-sized barrows was selected for blood sampling. Barrows were individually weighed and identified with numbered ear-tags to assure the same pig was used for each collection. A total of 8.5 mL of blood was collected and serum was allowed to clot on ice for at least 1.5 hr and centrifuged at 2,000×g for 13 min (4° C.) according to the instruction provided by BD (Franklin Lakes, N.J.).

Health status of each pen was accessed by fecal score on d 3, 5, 7, and 10 of the study using the following scoring method: 0=none; 1=mild; 2=substantial; 3=severe (Citation 1: Marquardt et al., 1999).

TABLE 9
Dietary Treatments

| | | | | # | Total # |
|---|---|---|---|---|---|
| Treatment | Additive | Inclusion Rate | # of Pens | pigs/pen | of pigs |
| A. Control | None | — | 16 | 27 | 432 |
| B. | Formulated yeast 1 | 2.0 lb/ton | 16 | 27 | 432 |
| C. | Formulated yeast 2¹ | 2.0 lb/ton | 16 | 27 | 432 |
| D. | Formulated yeast 3 | 2.0 lb/ton | 16 | 27 | 432 |
| E. | Formulated yeast 4 | 2.0 lb/ton | 16 | 27 | 432 |
| F. | Formulated yeast 4 + | Evo 4: 2.0 lb/ton + | | | |
| | SMBS ® with thiamin | 2.5 lb/ton FeedAid | 16 | 27 | 432 |
| | suppl. | and 0.3 lb/ton thiamin | | | |
| | | | | TOTAL | 2592 |

TABLE 10
Composition of Basal Diet

| | | | | |
|---|---|---|---|---|
| Ingredients | RES 9-15 | RES 15-25 | RES 25-40 | RES 40-55 |

Ingredients

| | | | | |
|---|---|---|---|---|
| corn 600 14% moisture | 648.90 | 881.04 | 944.58 | 998.48 |
| Soybean meal 46.2% | 200.00 | 432.00 | 534.00 | 497.00 |
| Crumble Basemix NHF | 951.09 | 475.00 | N/A | N/A |
| Premix2 | | | | |
| DOGS-Aurora SD | 100.00 | 100.00 | 400.00 | 400.00 |
| Choice White Grease | 30.00 | 40.00 | 60.00 | 50.00 |
| betaGRO | 6.00 | 3.00 | N/A | N/A |
| Lysine 98.5 | 6.50 | 7.40 | 10.80 | 10.80 |
| Methionine 99% DL | 3.75 | 3.50 | 2.50 | 2.30 |
| Threonine 98.5% | 2.55 | 2.80 | 2.85 | 2.80 |
| Tryptophan 98.5% | 0.71 | 0.56 | 0.57 | 0.62 |
| 21% Monocal | 10.10 | 19.90 | 5.70 | 2.10 |
| Limestone | 2.10 | 8.00 | 21.60 | 22.70 |
| Salt | 7.00 | 7.60 | 9.00 | 9.00 |
| Nursery VTM 3# | 3.00 | 3.00 | 3.00 | 3.00 |
| Optiphos 2000 | N/A | N/A | 0.50 | 0.70 |
| TBCC (Intellibond C) | N/A | N/A | 0.50 | 0.50 |
| ZnO 72% | 8.30 | 8.30 | N/A | N/A |
| Mecadox 2.5 | 20.0 | N/A | N/A | N/A |
| Aureomycin 90 (CTC) | N/A | 4.40 | 4.40 | N/A |
| Denagard 10 | N/A | 3.50 | N/A | N/A |
| Total Batch | 2000.00 | 2000.00 | 2,000.00 | 2,000.00 |

Nutrients

| | | | | |
|---|---|---|---|---|
| Mod ME, kcal/lb | 1514 | 1502 | 1500 | 1500 |
| SID Lysine, g/mcal | 4.19 | 4.08 | — | — |
| SID Thr/Ly, % | 0.63 | 0.62 | 0.61 | 0.61 |
| SID M + C/Lys, % | 0.58 | 0.58 | 0.56 | 0.56 |
| SID Try/Lys, % | 0.19 | 0.19 | 0.18 | 0.18 |
| SID Iso/Lys, % | 0.60 | 0.60 | 0.57 | 0.57 |
| SID Val/Lys, % | 0.65 | 0.65 | 0.65 | 0.65 |
| Protein, % | 23.31 | 22.66 | 22.52 | 21.81 |
| Lysine, % | 1.57 | 1.52 | 1.49 | 1.44 |
| SID Lysin % | 1.40 | 1.35 | 1.30 | 1.26 |
| Avail Phos Equil, % | 0.50 | 0.46 | 0.40 | 0.38 |
| Calcium, % | 0.67 | 0.69 | 0.78 | 0.75 |
| Anzl Calcium, % | 0.66 | 0.69 | 0.63 | 0.58 |
| Phosphorus, % | 0.74 | 0.74 | 0.55 | 0.50 |
| Lactose, % | 15.01 | 7.50 | — | — |
| Sodium, % | 0.41 | 0.30 | — | — |

TABLE 11
Feed Budget/Phase for Experimental Diets

| | | Feed Budget, | Approximate |
|---|---|---|---|
| Phase | Diet | lb/head | Days on Feed |
| Phase 1: 9-15 lb BW | Experimental | 6.0 | 7-10 |
| Phase 2: 15-25 lb BW | Experimental | 15.5 | 14 |
| Phase 3: 25-40 lb BW | Experimental | 22 | 14 |
| Phase 4: 40-55 lb BW | Experimental | 25 | 14 |

Pigs fed the combination of formulated yeast and sodium meta-bisulfite had improved (P<0.05) and a tendency for a reduced number of pigs removed due to mortality or sickness than the control pigs (Table 12). There were moderate improvements in performance for pigs fed the 3 variants of formulated yeast, however, not to the degree of the combination of yeast and sodium meta-bisulfite. Fecal scores tended to improve when combining the products together suggesting improved intestinal health. Both serum interferon alpha and IL-6 were reduced by feeding the combined product, further supporting the improvement in intestinal health which leads to improved feed conversion during the first two phases of the nursery period.

TABLE 12
2017-112N Formulated Yeast Formulation Study #3

| | | | | | | | | | | |
|---|---|---|---|---|---|---|---|---|---|---|
| | | | | | | | | | | P-Formu- |
| | | | | | | | | | | lated |
| | | | | | | | | | P-Control | yeast_4 vs. |
| | | | | | | Formu- | | | vs. Formu- | Formu- |
| | | Formu- | Formu- | Formu- | Formu- | alted | | | lated | lated |
| | | alted | alted | alted | alted | yeast_4 + | | P-value | yeast + | yeast + |
| Item | Control | yeast_1 | yeast_2 | yeast_3 | yeast_4 | SMBS | PSE | (over all) | SMBS | SMBS |

Treatments¹

| | | | | | | | | | | |
|---|---|---|---|---|---|---|---|---|---|---|
| # of Pens | 16 | 16 | 16 | 16 | 16 | 16 | | | | |
| # of Pigs | 432 | 432 | 432 | 432 | 432 | 432 | | | | |
| Initial | 10.5 | 10.5 | 10.5 | 10.4 | 10.5 | 10.5 | 0.1 | 0.97 | 0.60 | 0.63 |
| BW,² | | | | | | | | | | |
| lb | | | | | | | | | | |

Phase 1; 13 days

| | | | | | | | | | | |
|---|---|---|---|---|---|---|---|---|---|---|
| ADG, lb/day | 0.24 | 0.25 | 0.23 | 0.23 | 0.23 | 0.22 | 0.01 | 0.27 | 0.04 | 0.21 |
| ADFI, lb/day | 0.63^{a} | 0.62^{a} | 0.60^{ab} | 0.61^{ab} | 0.61^{ab} | 0.58^{b} | 0.01 | 0.003 | 0.0007 | 0.04 |
| F/G | 2.61 | 2.53 | 2.60 | 2.65 | 2.63 | 2.64 | 0.11 | 0.91 | 0.71 | 0.91 |
| BW end of | 13.7^{a} | 13.7^{a} | 13.6^{ab} | 13.6^{ab} | 13.6^{ab} | 13.4^{b} | 0.2 | 0.06 | 0.01 | 0.11 |
| Phase 1, lb | | | | | | | | | | |

Phase 2; 14 days

| | | | | | | | | | | |
|---|---|---|---|---|---|---|---|---|---|---|
| ADG, lb/day | 0.88 | 0.88 | 0.87 | 0.87 | 0.88 | 0.90 | 0.02 | 0.51 | 0.11 | 0.33 |
| ADFI, lb/day | 1.11 | 1.12 | 1.11 | 1.10 | 1.11 | 1.12 | 0.02 | 0.87 | 0.85 | 0.73 |
| F/G | 1.28 | 1.29 | 1.29 | 1.27 | 1.27 | 1.24 | 0.02 | 0.14 | 0.009 | 0.25 |
| BW end of | 26.0 | 26.1 | 25.8 | 25.7 | 25.8 | 26.0 | 0.5 | 0.89 | 0.85 | 0.57 |
| Phase 2, lb | | | | | | | | | | |

Phase 1 + 2; 27 days

| | | | | | | | | | | |
|---|---|---|---|---|---|---|---|---|---|---|
| ADG, lb/day | 0.56 | 0.56 | 0.55 | 0.56 | 0.56 | 0.57 | 0.01 | 0.78 | 0.59 | 0.38 |
| ADFI, lb/day | 0.88 | 0.88 | 0.86 | 0.86 | 0.86 | 0.86 | 0.01 | 0.59 | 0.15 | 0.67 |
| F/G | 1.56^{cd} | 1.55^{cd} | 1.56^{c} | 1.54^{cd} | 1.55^{cd} | 1.50^{d} | 0.02 | 0.11 | 0.007 | 0.07 |
| Non- | 1.62^{c} | 1.60^{cd} | 1.62^{c} | 1.58^{cd} | 1.60^{cd} | 1.53^{d} | 0.03 | 0.06 | 0.004 | 0.06 |
| adjusted | | | | | | | | | | |
| F/G | | | | | | | | | | |
| Removal, % | 5.56^{ab} | 5.32^{ab} | 6.25^{a} | 3.70^{ab} | 4.86^{ab} | 3.24^{b} | | 0.29 | 0.10 | 0.24 |
| Treated | 11.1 | 11.6 | 10.9 | 12.0 | 11.3 | 13.9 | | 0.75 | 0.25 | 0.29 |
| fallbacks, % | | | | | | | | | | |

Phase 3; 14 days

| | | | | | | | | | | |
|---|---|---|---|---|---|---|---|---|---|---|
| ADG, lb/day | 1.25 | 1.25 | 1.23 | 1.23 | 1.22 | 1.22 | 0.02 | 0.71 | 0.14 | 0.85 |
| ADFI, lb/day | 1.79 | 1.80 | 1.79 | 1.81 | 1.75 | 1.78 | 0.03 | 0.64 | 0.92 | 0.42 |
| F/G | 1.44 | 1.45 | 1.46 | 1.46 | 1.43 | 1.46 | 0.02 | 0.57 | 0.18 | 0.27 |
| BW end of | 43.4 | 43.6 | 43.1 | 43.1 | 42.9 | 43.1 | 0.6 | 0.80 | 0.55 | 0.74 |
| Phase 3, lb | | | | | | | | | | |

Phase 4; 14 days

| | | | | | | | | | | |
|---|---|---|---|---|---|---|---|---|---|---|
| ADG, lb/day | 1.42 | 1.39 | 1.42 | 1.44 | 1.41 | 1.41 | 0.03 | 0.54 | 0.64 | 0.76 |
| ADFI, lb/day | 2.25 | 2.26 | 2.27 | 2.27 | 2.26 | 2.26 | 0.05 | 1.00 | 0.92 | 0.98 |
| F/G | 1.59^{ab} | 1.62^{a} | 1.60^{ab} | 1.58^{b} | 1.60^{ab} | 1.61^{ab} | 0.01 | 0.07 | 0.18 | 0.55 |
| BW end of | 63.3 | 63.2 | 62.9 | 63.2 | 62.7 | 62.9 | 0.93 | 0.96 | 0.63 | 0.82 |
| Phase 4, lb | | | | | | | | | | |

Phase 3 + 4; 28 days

| | | | | | | | | | | |
|---|---|---|---|---|---|---|---|---|---|---|
| ADG, lb/day | 1.33 | 1.32 | 1.32 | 1.34 | 1.32 | 1.31 | 0.02 | 0.79 | 0.37 | 0.78 |
| ADFI, lb/day | 2.02 | 2.03 | 2.03 | 2.04 | 2.01 | 2.02 | 0.04 | 0.94 | 0.97 | 0.67 |
| F/G | 1.52 | 1.54 | 1.53 | 1.52 | 1.52 | 1.54 | 0.01 | 0.28 | 0.05 | 0.21 |
| Removal, % | 0.2 | 0.5 | 0.2 | 0.5 | 0.0 | 0.5 | | 0.80 | 0.56 | 0.16 |
| Treated | 0.2^{ab} | 0.9^{a} | 0.0^{b} | 0.0^{b} | 0.5^{ab} | 0.0^{b} | | 0.07 | 0.32 | 0.16 |
| fallbacks, % | | | | | | | | | | |

Overall; Phase 1-4; 55 days

| | | | | | | | | | | |
|---|---|---|---|---|---|---|---|---|---|---|
| ADG, lb/day | 0.95 | 0.95 | 0.94 | 0.95 | 0.94 | 0.95 | 0.02 | 0.96 | 0.79 | 0.76 |
| ADFI, lb/day | 1.45 | 1.46 | 1.45 | 1.46 | 1.44 | 1.45 | 0.02 | 0.98 | 0.79 | 0.81 |
| F/G | 1.53 | 1.54 | 1.54 | 1.53 | 1.53 | 1.53 | 0.01 | 0.33 | 0.92 | 0.95 |
| Removal, % | 5.8^{cd} | 5.8^{cd} | 6.5^{c} | 4.2^{cd} | 4.9^{cd} | 3.7^{d} | | 0.38 | 0.16 | 0.41 |
| Treated | 11.3 | 12.5 | 10.9 | 12.0 | 11.8 | 13.9 | | 0.80 | 0.29 | 0.39 |
| fallbacks, % | | | | | | | | | | |

Fecal score

| | | | | | | | | | | |
|---|---|---|---|---|---|---|---|---|---|---|
| Day 3 | 0.25 | 0.25 | 0.44 | 0.38 | 0.31 | 0.25 | 0.12 | 0.55 | 0.95 | 0.57 |
| Day 5 | 1.07 | 1.12 | 1.13 | 1.07 | 1.12 | 0.93 | 0.12 | 0.69 | 0.33 | 0.16 |
| Day 7 | 0.75 | 0.93 | 0.94 | 0.94 | 0.94 | 0.81 | 0.15 | 0.61 | 0.78 | 0.27 |
| Day 10 | 0.37 | 0.56 | 0.50 | 0.50 | 0.56 | 0.50 | 0.12 | 0.13 | 0.24 | 0.28 |
| Average of | 0.61 | 0.72 | 0.75 | 0.72 | 0.73 | 0.62 | 0.09 | 0.32 | 0.99 | 0.13 |
| fecal score | | | | | | | | | | |

Serum cytokines

| | | | | | | | | | | |
|---|---|---|---|---|---|---|---|---|---|---|
| End of | | | | | | | | | | |
| Phase 2³ | | | | | | | | | | |
| IFN-α, pg/mL | 3.36^{a} | 3.11^{a} | 2.98^{ab} | 2.89^{ab} | 2.34^{ab} | 1.76^{b} | 0.92 | 0.04 | 0.006 | 0.20 |
| IL-6, pg/mL | 7.89 | 7.18 | 8.01 | 7.57 | 7.19 | 6.62 | 4.29 | 0.21 | 0.03 | 0.27 |
| IL-8, pg/mL | 109.4 | 132.6 | 123.8 | 94.4 | 106.4 | 91.2 | 27.1 | 0.72 | 0.52 | 0.62 |
| IL-12, pg/mL | 812.8 | 693.1 | 821.5 | 1054.4 | 799.9 | 709.9 | 100.9 | 0.16 | 0.57 | 0.57 |

^{ab}Means without a common superscript differ (P < 0.05)
^{cd}Means without a common superscript ten to differ (P < 0.10)
¹Control = No Formulated yeast;
²Initial BW at weaning was used as covariate in all statistical models
³Values at weaning were used at covariate in statistical models

### Example 5: Effect of Formulated Yeast, Sodium Meta-Bisulfite and Bentonite Product VW on Weanling Pig Growth Performance

A total of 972 weanling pigs (27 pigs/pen, 36 total pens) were used to determine the impact of formulated yeast and sodium meta-bisulfite on weanling pig growth performance.

Upon arrival weanling pigs were sorted and weighted into pens, and blocked by weight into groups of 4 pens per weight block. Within block, pens were randomly assigned to one of four dietary treatments (1 control and 3 experimental diets; Table 13). This resulted in 12 pens for each dietary treatment for evaluation of growth performance, fecal scores, biological measurements, and health status. Diets containing testing products were made by mixing the testing products into the basal diet (Table 10).

This experiment was conducted for approximately 52 days in a 4-phase feeding program (Table 11) immediately after weaning. Feed was provided through the FeedLogic® system allowing collection of feed intake data by pen. Pigs were weighed at arrival, at each of the diet changes and at the end of the experiment to give five weight collections. Feed intake was collected over each of these periods and the data was used to calculate ADG, ADFI and F/G for the weight period, the combination of phases 1 and 2, phases 3 and 4, and for the entire experimental period.

Blood samples were collected at three time points: day 0, end of Phase 2, and Phase 4 from one barrow/pen (average size). A total of 8.5 mL blood was collected and serum was allowed to clot on ice for at least 1.5 hr and centrifuged at 2,000×g for 13 min (4° C.). Health status for each pen was determined by measuring pen fecal scores on day 3, 5, 7 and 10 using the following scoring method: 0=none; 1=mild; 2=substantial; 3=severe (Citation 1: Marquardt et al., 1999).

TABLE 13
Dietary Treatments

| | | Inclusion | | | Total # of |
|---|---|---|---|---|---|
| Treatment | Additive | Rate | # of Pens | # pigs/pen | pigs |
| 1. Control | None | — | 12 | 27 | 324 |
| 2. | Formulated | 2.0 lb/ton | 12 | 27 | 324 |
| | yeast A | Formulated | | | |
| | | yeast | | | |
| 3. | Formulated | 7.0 lb/ton | 12 | 27 | 324 |
| | yeast B¹ | Formulated | | | |
| | | yeast B | | | |
| | | | | TOTAL | 972 |

¹Formulated yeast A = Formulated yeast, Formulated yeast B = Formulated yeast + SMBS

Feeding formulated yeast alone to weanling pigs improved (P<0.05) ADG and F/G for phase 1 as well as for the combination of phases 1 and 2 (Table 14). Performance was further increased when cell wall was combined with sodium meta-bisulfite and bentonite. This improvement in the combined product was greater than the use of sodium meta-bisulfite and bentonite alone seen in previous examples. Performance was not further improved during phases 3 and 4, however, the combination did have an impact on overall growth rate for the study but not for feed conversion.

TABLE 14
Effects of Formulated Yeast on Growth Performance of Nursery Pigs.

| | | Formulated yeast | | | Probability, |
|---|---|---|---|---|---|
| Item | Control | A | B | SEM | P< |

BW

| | | | | | |
|---|---|---|---|---|---|
| Day 0 | 11.10 | 11.10 | 11.11 | 0.166 | 0.994 |
| Phase 1 | 11.18 | 11.37 | 11.43 | 0.199 | 0.449 |
| Phase 2 | 18.50^{b} | 18.92^{b} | 19.71^{a} | 0.285 | 0.005 |
| Phase 3 | 33.27^{b} | 33.96^{ab} | 34.22^{a} | 0.626 | 0.068 |
| Final BW | 50.04 | 50.81 | 51.15 | 0.849 | 0.160 |

Phase 1

| | | | | | |
|---|---|---|---|---|---|
| ADG | 0.01 | 0.03 | 0.04 | 0.011 | 0.169 |
| ADFI | 0.50^{b} | 0.52^{a} | 0.50^{b} | 0.007 | 0.025 |
| G:F | 0.01 | 0.06 | 0.08 | 0.020 | 0.133 |

Phase 2

| | | | | | |
|---|---|---|---|---|---|
| ADG | 0.54^{b} | 0.56^{b} | 0.61^{a} | 0.012 | 0.001 |
| ADFI | 0.86^{b} | 0.89^{ab} | 0.92^{a} | 0.016 | 0.071 |
| F/G | 1.61^{a} | 1.58^{a} | 1.50^{b} | 0.022 | 0.007 |

Phase 3

| | | | | | |
|---|---|---|---|---|---|
| ADG | 1.00^{ab} | 1.02^{a} | 0.99^{bc} | 0.022 | 0.001 |
| ADFI | 1.42^{b} | 1.48^{a} | 1.49^{a} | 0.035 | 0.023 |
| F/G | 1.43^{b} | 1.45^{b} | 1.52^{a} | 0.014 | 0.001 |

Phase 4

| | | | | | |
|---|---|---|---|---|---|
| ADG | 1.29 | 1.30 | 1.30 | 0.021 | 0.653 |
| ADFI | 1.93 | 1.95 | 1.97 | 0.033 | 0.429 |
| F/G | 1.50 | 1.51 | 1.52 | 0.012 | 0.478 |

Phase 1 & 2

| | | | | | |
|---|---|---|---|---|---|
| ADG | 0.34^{c} | 0.36^{b} | 0.40^{a} | 0.010 | 0.001 |
| ADFI | 0.73 | 0.75 | 0.76 | 0.011 | 0.149 |
| F/G | 2.18^{a} | 2.07^{ab} | 1.92^{c} | 0.040 | 0.001 |
| NonAdj F/G | 2.38^{a} | 2.10^{b} | 1.98^{b} | 0.081 | 0.011 |

Phase 3 & 4

| | | | | | |
|---|---|---|---|---|---|
| ADG | 1.13 | 1.15 | 1.13 | 0.020 | 0.165 |
| ADFI | 1.66^{b} | 1.70^{ab} | 1.72^{a} | 0.032 | 0.054 |
| F/G | 1.46^{b} | 1.48^{b} | 1.52^{a} | 0.010 | 0.001 |
| NonAdj F/G | 1.47^{c} | 1.49^{bc} | 1.53^{ab} | 0.013 | 0.005 |

Overall

| | | | | | |
|---|---|---|---|---|---|
| ADG | 0.78^{c} | 0.81^{ab} | 0.81^{a} | 0.016 | 0.015 |
| ADFI | 1.25^{b} | 1.29^{a} | 1.30^{a} | 0.023 | 0.018 |
| F/G | 1.60 | 1.60 | 1.60 | 0.010 | 0.150 |
| NonAdj F/G | 1.63 | 1.61 | 1.62 | 0.015 | 0.140 |

¹Formulated yeast A = Formulated yeast, Formulated yeast B = Formulated yeast + Feed Aid,

### Example 6: Effect of Yeast Cell Wall, Sodium Meta-Bisulfite on Weanling Pig Growth Performance

A total of 972 weanling pigs (27 pigs/pen, 36 total pens) were used to determine the effect of feeding yeast cell wall, sodium meta-bisulfite and bentonite clay to weanling pigs. At arrival, weanling pigs were sorted into pens and weighed on pen basis. Pens were ranked and blocked by weight, and pens assigned to 1 of 4 dietary treatments (1 control and 3 experimental diets; (Table 15) in a randomized complete block design. This will result in 12 pens for each dietary treatment for evaluation of growth performance, fecal scores, biological measurements, and health status. Pigs will be weighed on pen basis upon arrival, and by the time the majority of pens complete each ration. Feed leftover of each pen was measured on the same days when pigs are weighed. Data from the FeedLogic® system was saved for every feeding activity, and used to calculate ADG, ADFI and F:G of each pen.

Blood samples were collected on day 0, end of Phase 2, and Phase 4 from one barrow/pen (average size). 8.5 mL of blood was collected and serum was allowed to clot on ice for at least 1.5 hr and centrifuged at 2,000×g for 13 min (4° C.). Health status of each pen was recorded on each pen by accessing fecal score with the following scale on days 3, 5, 7, and 10:0=none; 1=mild; 2=substantial; 3=severe (Citation 1: Marquardt et al., 1999).

TABLE 15
Dietary Treatments

| | | Inclusion | # | # | Total # of |
|---|---|---|---|---|---|
| Treatment | Additive | Rate | of Pens | Pigs/Pen | Pigs |
| 1. Control | None | — | 12 | 27 | 324 |
| 2. | Formulated | 2.0 lb/ton | 12 | 27 | 324 |
| | yeast A | Formulated | | | |
| | | yeast | | | |
| 3. | Formulated | 5.7 lb/ton | 12 | 27 | 324 |
| | yeast D¹ | Formulated | | | |
| | | yeast D | | | |
| | | | | Total | 972 |

¹Formulated yeast A = Formulated yeast, Formulated yeast D = Cell wall + SMBS

Pigs fed yeast cell wall had improved (P<0.05) ADG and F/G compared to control fed pigs for the combined phase 1 and 2 periods as well as for the entire experiment (Table 16). The additional sodium meta-bisulfate and bentonite to the diets further improved (P<0.05) the growth rate and feed conversion to the pigs fed yeast cell wall. These data suggest that the combination further improves performance and offers an unique combination for improving health and performance of newly weaned pigs.

TABLE 16
Effects of Formulated Yeast on Growth Performance of Nursery Pigs

| | Formulated Yeast¹ | | | Probability, |
|---|---|---|---|---|
| Control | A | D | SEM | P< |

BW

| | | | | | |
|---|---|---|---|---|---|
| Day 0 | 11.00 | 10.99 | 10.99 | 0.252 | 0.999 |
| Phase 1 | 11.29 | 11.16 | 11.46 | 0.326 | 0.212 |
| Phase 2 | 19.54^{c} | 20.27^{b} | 21.06^{a} | 0.525 | 0.001 |
| Phase 3 | 34.03^{c} | 34.87^{cb} | 36.33^{a} | 0.823 | 0.001 |
| Final BW | 51.57^{b} | 53.48^{a} | 54.52^{a} | 1.259 | 0.003 |

Phase 1

| | | | | | |
|---|---|---|---|---|---|
| ADG | 0.03^{ab} | 0.02b | 0.06^{a} | 0.013 | 0.049 |
| ADFI | 0.49 | 0.48 | 0.50 | 0.009 | 0.675 |
| G:F | 0.07^{ab} | 0.03^{b} | 0.11^{a} | 0.026 | 0.044 |

Phase 2

| | | | | | |
|---|---|---|---|---|---|
| ADG | 0.58^{c} | 0.64^{b} | 0.68^{a} | 0.018 | 0.001 |
| ADFI | 0.88^{b} | 0.92^{a} | 0.93^{a} | 0.023 | 0.029 |
| F/G | 1.53^{a} | 1.44^{b} | 1.38^{b} | 0.029 | 0.004 |

Phase 3

| | | | | | |
|---|---|---|---|---|---|
| ADG | 1.03^{c} | 1.04^{bc} | 1.09^{a} | 0.025 | 0.013 |
| ADFI | 1.37^{b} | 1.42^{a} | 1.46^{a} | 0.040 | 0.011 |
| F/G | 1.34^{ab} | 1.37^{a} | 1.34^{ab} | 0.014 | 0.034 |

Phase 4

| | | | | | |
|---|---|---|---|---|---|
| ADG | 1.25 | 1.32 | 1.30 | 0.034 | 0.170 |
| ADFI | 1.94 | 2.03 | 1.98 | 0.054 | 0.255 |
| F/G | 1.55 | 1.55 | 1.52 | 0.013 | 0.141 |

Phase 1 & 2

| | | | | | |
|---|---|---|---|---|---|
| ADG | 0.38^{c} | 0.41^{b} | 0.45^{a} | 0.015 | 0.001 |
| ADFI | 0.73^{b} | 0.76^{ab} | 0.77^{a} | 0.016 | 0.030 |
| F/G | 1.97^{a} | 1.87^{ab} | 1.74^{c} | 0.044 | 0.002 |
| Adj F/G | 2.07^{a} | 1.99^{ab} | 1.81^{b} | 0.068 | 0.044 |

Phase 3 & 4

| | | | | | |
|---|---|---|---|---|---|
| ADG | 1.14^{b} | 1.18^{ab} | 1.20^{a} | 0.028 | 0.054 |
| ADFI | 1.66 | 1.73 | 1.72 | 0.044 | 0.147 |
| F/G | 1.46^{ab} | 1.47^{a} | 1.44^{b} | 0.011 | 0.036 |
| Adj F/G | 1.46^{ab} | 1.48^{a} | 1.44^{b} | 0.012 | 0.011 |

Overall

| | | | | | |
|---|---|---|---|---|---|
| ADG | 0.80^{c} | 0.83^{b} | 0.86^{a} | 0.020 | 0.001 |
| ADFI | 1.24^{b} | 1.29^{a} | 1.30^{a} | 0.030 | 0.009 |
| F/G | 1.56^{a} | 1.55^{a} | 1.51^{b} | 0.010 | 0.001 |
| Adj F/G | 1.58^{a} | 1.59^{a} | 1.53^{b} | 0.014 | 0.004 |

^{a-c}within a row, means without a common superscript differ (P < 0.05)
¹Formulated yeast A = Formulated yeast, Formulated yeast D = Cell wall + SMBS.

### Example 7: Effect of Sodium Metabisulfite Vs SMBS+Formulated Yeast on Post-Weaning Pig Growth Performance

A total of 972 pigs were used to determine the effect of sodium metabisulfite (SMBS) or SMBS plus formulated yeast on weanling pig growth performance. Upon arrival pigs were sorted to pens with 27 pigs per pen. Pigs were weighed on pen basis. Pens within block were randomly assigned to one of 3 dietary treatments (1 control and 2 experimental diets; Table 17) in a randomized complete block design. This will result in 12 pens for each treatment for evaluation of growth performance, fecal scores, and health status.

TABLE 17
Dietary treatments

| | Inclusion | # of | # | Total # |
|---|---|---|---|---|
| Treatment | Rate | Pens | pigs/pen | of pigs |
| 1. Control | — | 12 | 27 | 324 |
| 2. Formulated | 5.0 lb/ton | 12 | 27 | 324 |
| yeast + SMBS | | | | |
| 3. SMBS | 4.0 lb/ton | 12 | 27 | 324 |
| | | | TOTAL | 972 |

This experiment was conducted for approximately 22 days in a 2-phase feeding program (Table 18) immediately after weaning. Feed was provided through the FeedLogic® system allowing collection of feed intake data by pen. The composition of each experimental diet is shown in Table 19.

TABLE 18
Feed budget/phase for experimental diets

| | | Feed Budget, | Approximate |
|---|---|---|---|
| Phase | Diet | lb/head | Days on Feed |
| Phase 1: 9-15 lb BW | Experimental | 4.0 | 7-10 |
| Phase 2: 15-25 lb BW | Experimental | 15.5 | 14 |

TABLE 19
Composition of basal diet

| Ingredients | RES 9-15¹ | RES 15-25¹ |
|---|---|---|
| corn 600 14% moisture | 678.98 | 894.42 |
| Soybean meal 46.2% | 200.00 | 432.00 |
| Crumble Basemix NQ | 951.09 | 475.00 |
| Premix2 | | |
| DOGS-Aurora SD | 100.00 | 100.00 |
| Choice White Grease | 12.00 | 31.00 |
| betaGRO | 6.00 | 3.00 |
| Lysine 98.5 | 6.50 | 7.40 |
| Methionine 99% DL | 3.75 | 3.50 |
| Threonine 98.5% | 2.55 | 2.80 |
| Tryptophan 98.5% | 0.71 | 0.56 |
| 21% Monocal | 10.10 | 19.90 |
| Limestone | 2.10 | 8.00 |
| Salt | 7.00 | 7.60 |
| Nursery VTM 3# | 3.00 | 3.00 |
| Optiphos 2000 | N/A | N/A |
| TBCC (Intellibond C) | N/A | N/A |
| ZnO 72% | 8.30 | 8.30 |
| Aureomycin 90 (CTC) | 4.40 | N/A |
| Denagard 10 | 3.50 | 3.50 |
| Micro-tracer, 10 g/ton | 0.02 | 0.02 |
| Total Batch | 2000.00 | 2000.00 |
| Mod ME, kcal/lb | 1514 | 1502 |
| SID Lysine, g/mcal | 4.19 | 4.08 |
| SID Thr/Ly, % | 0.63 | 0.62 |
| SID M+C/Lys, % | 0.58 | 0.58 |
| SID Try/Lys, % | 0.19 | 0.19 |
| SID Iso/Lys, % | 0.60 | 0.60 |
| SID Val/Lys, % | 0.65 | 0.65 |
| Protein, % | 23.31 | 22.66 |
| Lysine, % | 1.57 | 1.52 |
| SID Lysin, % e | 1.40 | 1.35 |
| Avail Phos Equil, % | 0.50 | 0.46 |
| Calcium, % | 0.67 | 0.69 |
| Anzl Calcium, % | 0.66 | 0.69 |
| Phosphorus, % | 0.74 | 0.74 |
| Lactose, % | 15.01 | 7.50 |
| Sodium, % | 0.41 | 0.30 |

Pigs fed formulated yeast+SMBS had improved average daily gain (P<0.01) compared to pigs fed the control diet or SMBS alone in Phase 1 (Table 20). Feed intake was similar between all three treatments, resulting in improved (P<0.01) F/G for pigs fed formulated yeast plus SMBS compared to controls or SMBS. Both treatments improved (P<0.05) averaged daily gain and feed intake, however the combination resulted in a greater improvement than when feeding SMBS alone. Feed conversion was improved for both treatments compared to the control fed pigs, although feeding the combination resulted in a larger improvement in feed conversion than in pigs fed SMBS alone. The combined phase 1 and 2 performance was improved (P<0.01) for both treatments, however, pigs fed the formulated yeast plus SMBS treatment had a 3.4% better response than SMBS alone.

TABLE 20
Formulated yeast plus SMBS compared with SMBS

| | Treatment¹ | | | | |
|---|---|---|---|---|---|
| | | Formulated | | | |
| Item | Control | Yeast + SMBS | SMBS | PSE | P-value |
| # of Pens | 12 | 12 | 12 | | |
| # of Pigs | 324 | 324 | 324 | | |
| Start BW, lb | 11.9 | 11.8 | 11.8 | 0.2 | 0.99 |
| Phase 1; 8 days | | | | | |
| ADG, lb/day | 0.17^{cd} | 0.20^{c} | 0.16^{d} | 0.01 | 0.12 |
| ADFI, lb/day | 0.46 | 0.46 | 0.46 | 0.01 | 0.90 |
| G:F | 0.38^{cd} | 0.43^{c} | 0.35^{d} | 0.02 | 0.07 |
| BW end of | 13.5 | 13.7 | 13.4 | 0.1 | 0.14 |
| Phase 1, lb | | | | | |
| Phase 2; 14 days | | | | | |
| ADG, lb/day | 0.67^{a} | 0.77^{b} | 0.76^{b} | 0.02 | <.0001 |
| ADFI, lb/day | 0.92^{a} | 0.98^{b} | 0.96^{ab} | 0.02 | 0.04 |
| F:G | 1.38^{a} | 1.27^{b} | 1.27^{b} | 0.02 | <.0001 |
| BW end of | 22.9^{a} | 24.6^{b} | 24.1^{b} | 0.3 | <.0001 |
| Phase 2, lb | | | | | |
| Phase 1 + 2; 22 days | | | | | |
| ADG, lb/day | 0.49^{a} | 0.56 | 0.54 | 0.01 | 0.0001 |
| | | (+14.3%)^{b} | (+10.2%)^{b} | | |
| ADFI, lb/day | 0.75^{c} | 0.79^{d} | 0.78^{cd} | 0.01 | 0.08 |
| F:G | 1.55^{a} | 1.40 | 1.45 | 0.02 | <.0001 |
| | | (−9.7%)^{b} | (−6.5%)^{b} | | |
| Total removal, % | 2.2 | 1.9 | 2.2 | | 0.95 |
| Mortality, % | 0.0 | 0.3 | 0.6 | | 0.37 |

Citation 1: Marquardt, R. R., L. Z. Jin, J. W. Kim, L. Fang, A. A. Frohlich and S. K. Baidoo. 1999. Passive protective effect of egg-yolk antibodies against enterotoxigenic Escherichia coli K88+ infection in neonatal and early-weaned piglets. FEMS Immunol Med Microbiol 23: 283-288

What is claimed is:

1. A feed additive composition for non-human animals, the composition comprising:
a. formulated yeast; and
b. sodium metabisulfite.

2. The composition of claim 1, wherein the composition comprises between about 0.5 lb/ton and about 10.0 lb/ton of formulated yeast.

3. The composition of claim 2, wherein the composition comprises between about 2.0 lb/ton and about 7.0 lb/ton of formulated yeast.

4. The composition of claim 1, wherein the composition comprises between about 1.0 lb/ton and about 20.0 lb/ton of sodium metabisulfite.

5. The composition of claim 4, wherein the composition comprises between about 2.0 lb/ton and about 15.0 lb/ton of sodium metabisulfite.

6. The composition claim 1, wherein the composition further comprises thiamin.

7. The composition of claim 6, wherein the composition comprises between about 0 ppm to 20 ppm thiamin.

8. The composition of claim 7, wherein the composition comprises between about 2 ppm and about 10 ppm thiamin.

9. A method for improving growth in non-human animals, the method comprising: supplementing a normal feed or a basal feed with a feed additive composition comprising:
a. formulated yeast;
b. sodium metabisulfite; and
c. optionally thiamin.

10. The method of claim 9, wherein the feed additive composition comprises between about 1.0 lb/ton and about 5.0 lb/ton of formulated yeast.

11. The method of claim 9, wherein the feed additive composition comprises between about 1.0 lb/ton and about 20.0 lb/ton of sodium metabisulfite.

12. The method of claim 9, wherein the feed additive composition comprises between about 0 ppm to 20 ppm of thiamin.

13. The method of claim 9, wherein the feed additive composition is formulated with the normal animal feed or basal feed for oral administration to the non-human animal.

14. The method of claim 13, wherein the combination of the normal animal feed or basal feed and feed additive composition is administered to the non-human animal at least once per day.

15. The method of claim 9, wherein administering the feed additive composition with the normal animal feed or basal feed to the non-human animal improves growth as compared to a control group, formulated yeast alone and sodium metabisulfite alone; wherein improved growth is measure by an increase in the average daily weight gain (ADG), the average daily food intake (ADFI), improves overall body weight, and the ratio F/G; and
wherein F/G is defined as the ADFI/ADG.

16. The method of claim 9, wherein the non-human animal is selected from a group comprising growing pigs, calves, foals, kids (goats), lambs, cria, chicks, poults, ducklings, puppies, kittens, or combinations thereof.

17. A method for improving health in non-human animals, the method comprising: supplementing a normal animal feed or a basal feed with a feed additive composition comprising:
a. formulated yeast;
b. sodium metabisulfite; and
c. optionally thiamin.

18. The method of claim 17, wherein the feed additive composition comprises between about 1.0 lb/ton and about 5.0 lb/ton of formulated yeast.

19. The method of claim 17, wherein the feed additive composition comprises between about 1.0 lb/ton and about 20.0 lb/ton of sodium metabisulfite.

20. The method of claim 17, wherein the feed additive composition comprises between about 0 to 20 ppm of thiamin.

21. The method of claim 17, wherein the feed additive composition is formulated with the normal feed or basal feed for oral administration to the non-human animal.

22. The method of claim 21, wherein the combination of the normal animal feed or basal feed and feed additive composition is administered at least once per day.

23. The method of claim 17, wherein administering the feed additive with normal animal feed or basal feed to the non-human animal improves health; wherein improved health is measured by the reduction of incidences of diarrhea, the reduction of days of diarrhea, a decrease in mortality, a decrease in morbidity, a reduction of cytokine panel measuring TNF-alpha, a reduction immunocrit (increased IgG) levels, or combinations thereof as compared to a control group.

24. The composition of claim 17, wherein the non-human animal is selected from a group comprising growing pigs, calves, foals, kids (goats), lambs, cria, chicks, poults, ducklings, puppies, kittens, or combinations thereof.

25. A method for improving intestinal health and the reduction of microbial pathogens in non-human animals, the method comprising: supplementing a normal animal feed or a basal feed with a feed additive composition comprising:
a. formulated yeast;
b. sodium metabisulfite; and
c. optionally thiamin.

26. The method of claim 25, wherein the feed additive composition comprises between about 1.0 lb/ton and about 5.0 lb/ton of formulated yeast.

27. The method of claim 25, wherein the feed additive composition comprises between about 1.0 lb/ton to about 20.0 lb/ton of sodium metabisulfite.

28. The method of claim 25, wherein the feed additive composition comprises between about 0 and about 20 ppm thiamin.

29. The method of claim 25, wherein the feed additive composition is formulated with the normal feed or basal feed for oral administration to the non-human animal.

30. The method of claim 29, wherein the combination of the normal feed or the basal feed and feed additive composition is administered at least once per day to the non-human animal.

31. The method of claim 25, wherein administering the feed additive with normal animal feed or basal feed reduces the number of pathogens in the non-human animal as compared to a control group.

32. The method of claim 31, wherein the pathogens are selected from a group comprising comprise E. Coli, E. Coli F18, E. Coli K88, salmonella, clostridium perfringens, Enterotoxigenic Escherichia coli (ETEC), and S. suis.

33. The method of claim 32, wherein administering the feed additive with normal animal feed or basal feed reduces inflammation caused by pathogens in the non-human animal as compared to the control group.

34. The method of claim 25, wherein the reduction of pathogens is measured by a reduction in serum kinase levels of IFN-α, IL-6, IL-8, and IL-12.

35. The composition of claim 25, wherein the non-human animal is selected from a group comprising growing pigs, calves, foals, kids (goats), lambs, cria, chicks, poults, ducklings, puppies, kittens, or combinations thereof.
